(19) United States
(12) Patent Application Publication
(10) Pub. No.: US 2004/0096880 A1
(43) Pub. Date: May 20, 2004

(54) Compositions and methods for the treatment of diseases exhibiting protein misassembly and aggregation

(76) Inventors: Eric B. Kmiec, Landenberg, PA (US); Hetal Parekh-Olmedo, Deptford, NJ (US)

Correspondence Address: FISH & NEAVE, 1251 AVENUE OF THE AMERICAS, 50TH FLOOR, NEW YORK, NY, 10020-1105, US

(21) Appl. No.: 10/638,060

(22) Filed: Aug. 7, 2003

Related U.S. Application Data
(63) Continuation-in-part of application No. 10/215,432, filed on Aug. 7, 2002.
(60) Provisional application No. 60/402,198, filed on Aug. 7, 2002.
(60) Provisional application No. 60/337,219, filed on Dec. 4, 2001.
(60) Provisional application No. 60/310,770, filed on Aug. 8, 2001.
(60) Provisional application No. 60/310,889, filed on Aug. 8, 2001.
(60) Provisional application No. 60/310,757, filed on Aug. 7, 2001.

(51) Int. Cl. C12Q 1/68

(52) U.S. Cl. 435/6

(57) ABSTRACT

Compounds, compositions, and pharmaceutical compositions comprising oligonucleotides capable of disrupting protein aggregations that are characteristic of disorders of protein assembly are described, as are in vitro and in vivo methods for identifying usch oligonucleotides, and methods for treating such disorders by administration of such compositions.

### CROSS-REFERENCE TO RELATED APPLICATION

This application is a continuation-in-part of U.S. application Ser. No. 10/215,432, filed Aug. 7, 2002, which claims the benefit of U.S. provisional application No. 60/337,219, filed Dec. 4, 2001, 60/310,889, filed Aug. 8, 2001, 60/310,770, filed Aug. 8, 2001, and 60/310,757, filed Aug. 7, 2002; and also claims the benefit of U.S. provisional application No. 60/402,198, filed Aug. 7, 2002, the disclosures of which are incorporated herein by reference in their entireties.

### FIELD OF THE INVENTION

The present invention is in the field of cell biology, and relates to compounds, compositions, and pharmaceutical compositions capable of disrupting pathological protein aggregates, to methods for identifying such compounds, compositions, and pharmaceutical compositions, and to methods for treating diseases that are characterized by protein misassembly and aggregation.

### BACKGROUND OF THE INVENTION

The misassembly and aggregation of proteins that are normally soluble appears to be responsible for a wide variety of diseases. These include inherited neurodegenerative diseases, such as Huntington's disease (“HD”), Alzheimer's disease, cystic fibrosis, amyotrophic lateral sclerosis and Parkinson's disease; inherited blood disorders; infectious prion diseases, such as scrapie of sheep and goats, bovine spongiform encephalopathy of cattle, and kuru, Creutzfeldt-Jakob Disease (CJD), Gerstmann-Sträussler-Sheinker Disease, and fatal familial insomnia of humans.

Huntington's disease belongs to a family of neurodegenerative diseases, including spinobulbar muscular atrophy, spinocerebellar ataxia types 1, 2, 3, 6, and 7, and dentatorubral-pallidoluysian atrophy that in most cases are dominantly inherited and that are characterized by mutations in which CAG trinucleotide sequences are expanded, causing the translation of proteins with abnormally long polyglutamine tracts. See, e.g., Carmichael et al., Proc. Nat'l Acad. Sci. USA, 97:9701-9705 (2000). Although the diseases result in neurodegeneration at different locations, they all share the same basic pathological cause: the formation of intracellular protein aggregates within the vulnerable neurons and the consequent loss of function of those neurons.

In the case of Huntington's disease, the protein aggregates are composed of ubiquitinated, N-terminal fragments of “Huntingtin”, the protein encoded by the gene associated with the disease. DiFiglia et al., Science, 277:1990-93 (1997). These fragments include the region of polyglutamine expansion that is associated with the disease-causing form of the protein. Both formation of these aggregates in mammalian cell models and cell death have been found to be reduced in the presence of a bacterial chaperone, GroEL, and HSP104, a yeast heat shock protein, suggesting that there is a causal link between aggregation of the protein and disease pathology. Carmichael et al., Proc. Nat'l Acad. Sci. USA, 97:9701-9705 (2000).

Aggregation of proteins within a cell has recently been shown directly to impair the function of the ubiquitin-proteasome system responsible for degrading misfolded, unassembled or damaged proteins that could potentially form toxic aggregates within a cell. Bence et al., Science, 292:1552-55 (2001). Such an impairment may create a positive feedback mechanism whereby the increased levels of aggregated proteins inhibit the very system responsible for degrading those proteins and could result in the precipitous loss of cell function characteristic of many neurodegenerative diseases. Consistent with this view, direct inhibition of proteasome activity results in the accumulation of mutant Huntingtin fragments in aggresome-like inclusion bodies. Waelter et al., Mol. Biol. Cell, 12:1393-1407 (2001).

Alzheimer's disease is another disorder in which protein aggregation appears to be responsible, at least in part, for the pathology of the disease. Amyloid isolated from the brain tissue of Alzheimer's disease patients consists mainly of proteins from the family designated “Aβ” (for Amyloid plaque of β secondary structure). Koo et al., Proc. Nat'l Acad. Sci. USA, 96:9989-90 (1999). A minor component of the amyloid plaques, Aβ₄₂, contains a sequence that can form unusually stable and ordered fibrils. This component may be responsible for “nucleating” fibril formation. Although Aβ₄₂ levels are not elevated in the most common, sporadic forms of Alzheimer's disease, they do increase in patients with mutations in presenilin 1, presenilin 2 and amyloid beta-protein precursor that are linked to familial Alzheimer's disease. Scheuner et al., Nature Med., 2:864-70 (1996). Increased levels of Aβ₄₂ may arise from changes in the cleavage pattern of the β-amyloid precursor proteins (“APP”) (Sisodia, Science, 289:2296-97 (2000)) or by decreased levels of degradation of Aβ₄₂ itself. Iwata et al., Science, 292:1550-52 (2001).

Parkinson's disease is another common neurodegenerative motor disorder that is believed to result from improper protein interactions. Although environmental factors had long been thought to be responsible for the condition, genetic factors have now been implicated as well. See Cole et al., Neuromolecular Med., 1:95-109 (2002). In particular, mutations in the gene encoding the presynaptic protein, α-synuclein, may be responsible for the accumulation of this protein in Lewy bodies, the neuropathological hallmark of Parkinson's disease.

Prion diseases also appear to result from the misfolding and aggregation of specific proteins. PrP^{C} is the normal form of the prion protein and is encoded by a single-copy gene in mammals. Basler et al., Cell, 46:417-28 (1986). When expressed, the protein is generally found on the surface of neuronal cells. Prion diseases are believed to result from the conversion of the normal, PrP^{C}-form of the prion protein to an insoluble, disease-causing form, PrP^{Sc}. Although the amino acid sequences of the two forms are identical, the proteins appear to differ in conformation. Pan et al., Proc. Nat'l Acad. Sci. USA, 90:10962-66 (1993). PrP^{Sc} appears to be involved both in the transmission of prion diseases and in their pathogenesis. As noted above, prion diseases include scrapie of sheep and goats; bovine spongiform encephalopathy of cattle; and kuru, Creutzfeldt-Jakob Disease, Gerstmann-Sträussler-Sheinker Disease, and fatal familial insomnia of humans. See Prusiner et al., U.S. Pat. No. 6,214,366.

Protein misassembly and aggregation have also been implicated in the syndromes known as familial amyloid polyneuropathy and senile systemic amyloidosis. Kelly, Curr. Opin. Struct. Biol., 6:11-17 (1996). Amyloid fibrils form in various tissues from mutated forms of transthyretin, a tetrameric protein involved in the transport of thyroxine and retinol. Jacobson et al., Adv. Hum. Genet., 20:69-123 (1991). The mutations appear to destabilize the tetrameric structure and allow an amyloidogenic intermediate to form under the low pH conditions found in the lysosome. Miroy et al., Proc. Nat'l Acad. Sci. USA 93:15051-56 (1996). The wild-type form of the protein remains tetrameric and nonamyloidogenic under these conditions.

Many other diseases are thought to involve protein misfolding, misassembly, and/or aggregation. Such diseases (and the implicated protein) include: amyotrophic lateral sclerosis (superoxide dismutase), Pick's disease (tau protein in Pick bodies), diabetes type II (amylin), multiple myeloma-plasma cell dyscrasias (IgG light chain), medullary carcinoma of the thyroid (procalcitonin), chronic renal failure (β₂-microglobulin), congestive heart failure (atrial natriuretic factor), chronic inflammation (serum amyloid A), atherosclerosis (apoA1), familial amyloidosis (gelsolin). See Prusiner et al., U.S. Pat. No. 6,214,366.

Although the misfolding and aggregation of proteins has now been implicated in the pathogenesis of many diseases, few treatments are currently available to reverse the aggregation process and to ameliorate the pathological conditions. Several experimental results suggest, however, that reversing aggregation will prove therapeutically valuable.

For example, several dendritic polycations have recently been found to eliminate scrapie prion precursor protein from cultured scrapie-infected neuroblastoma cells, but the mechanism of this process is unclear. Prusiner et al., U.S. Pat. No. 6,214,366.

A small-molecule inhibitor of the binding of serum amyloid P component (“SAP”) to fibrils has recently been identified and suggested for use in the treatment of human amyloidosis. Pepys et al., Nature, 417:254-59 (2002). Treatment of seven human systemic amyloidosis patients with a small-molecule inhibitor of SAP binding to amyloid fibrils resulted in a substantial decrease in levels of circulating SAP. Pepys et al., Nature, 417:254-59 (2002). Methods to screen for additional inhibitors of SAP binding to amyloid fibrils have been disclosed. Pepys et al., U.S. Pat. No. 6,126,918.

In vitro biophysical experiments have shown that the binding of ligands of transthyretin to the protein can stabilize the tetrameric form of the protein and inhibit the formation of amyloid. Miroy et al., Proc. Nat'l Acad. Sci. USA, 93:15051-56 (1996).

A bivalent suppressor protein containing polyglutamine segments separated by a spacer segment derived from a helical region of the TATA-binding protein inhibits the aggregation of a protein containing an expanded polyglutamine repeat sequence in cultured cells. Karantsev et al., Nature Genetics, 30:367-76 (2002). This same suppressor construct inhibits adult lethality and neuron degeneration when expressed in a transgenetic Drosophila model. Id.

Transglutaminase inhibitors, such as cystamine and monodansyl cadaverine, have been used to suppress aggregate formation and apoptotic cell death in cultured cells that express proteins containing expanded polyglutamine segments. Tsuji, U.S. Pat. No. 6,355,690.

Fusaric acid and picolinic acid have been used to prevent the zinc-dependent polymerization and fibril formation of βA₁₋₄₀ amyloid peptide. Douglas et al., U.S. Patent Publication No. US 2002/0037908 A1. These same agents also cause the release of β-amyloid protein from brain slices of post-mortem Alzheimer's disease patients. Id.

The above findings indicate that agents able to inhibit the formation of protein aggregates or disrupt the structure of already formed aggregates may be useful in treating the diseases caused by protein misassembly and aggregation. U.S. Pat. No. 6,420,122 describes in vitro methods for identifying additional agents useful for effecting disruption of protein aggregates in cells.

Given the large number of such diseases and the severity of their effects on individuals and on society, there is a clear need to develop new and effective therapeutic approaches.

### SUMMARY OF THE INVENTION

The present invention is based upon the unexpected discovery that oligonucleotides unrelated in sequence to that of the nucleic acid which encodes the protein aggregant can be effective in disrupting or preventing aggregation in disorders of protein assembly.

In a first aspect, the invention provides a method for identifying oligonucleotides that are effective to prevent, reduce or disrupt aggregation of a protein aggregant in a cell.

The method comprises identifying, from a plurality of oligonucleotide species differing in sequence, those oligonucleotide species that are effective to prevent, reduce, or disrupt aggregation of a protein aggregant in a cell, by introducing each of a plurality of oligonucleotide species of disparate sequence separately into cells that have or are likely to develop protein aggregates, and identifying oligonucleotide species that are effective at preventing, reducing, or disrupting aggregation and/or increasing cell survival.

The protein aggregant may be selected, among others, from the group consisting of huntingtin (htt), Aβ, tau, α-synuclein, atropin-1, ataxin-1, ataxin-2, ataxin-3, ataxin-7, alpha 1A, PrP^{sc}, transthyretin, superoxide dismutase, amylin, IgG light chain, procalcitonin, β₂-microglobulin, atrial natriuretic factor, serum amyloid A, apoA1, and gelsolin.

The oligonucleotides can be at least 4 nt in length, typically at least 6 nt in length, and may usefully be at least 9 nt, 25 nt, even 30 nt or more in length; in some embodiments, the oligonucleotides can be at laest 35 nt, 40 nt, 45 nt, even 50 nt in length or more. The oligonucleotides may usefully and typically have a modification, such as one or more phosphorothioate linkages or 2′-OMe analogues. In some embodiments, the oligonucleotides are nonidentical and noncomplementary in sequence to any portion of 10 or more contiguous nucleotides of the nucleic acid that encodes the protein aggregant. In other embodiments, the oligonucleotides may bear sequence complementarity or identity, at least in part, to a portion of the nucleic acid sequence that encodes the protein aggregant. In screening embodiments, the plurality of oligonucleotide species are typically nonidentical to one another.

In one series of embodiments, the oligonucleotides are introduced into the cells in vitro, typically by a transfection method selected from the group consisting of passive transfection, chemical transfection and mechanical transfection.

Either or both of the oligonucleotide and protein aggregant can be detectably labeled. In embodiments in which the aggregant is labeled, typically, although not necessarily, the label is recombinantly fused to the aggregant. Such labels may, for example, be a polypeptide comprising a GFP-like chromophore.

In other embodiments, the oligonucleotides are introduced into the cells in vivo.

In a second aspect, the invention provides a method of treating a subject having a disorder of protein assembly. The method comprises administering an effective amount of a composition that comprises at least one oligonucleotide species that prevents, reduces, or disrupts protein aggregation, optionally in admixture with a pharmaceutically acceptable carrier or excipient.

In most embodiments, the oligonucleotide is at least 4 nt in length, typically at least 6 nt in length, and may usefully be at least 9 nt, 25 nt, even 30 nt or more in length; in some embodiments, the oligonucleotides can be at least 35 nt, 40 nt, 45 nt, even 50 nt in length or more. The oligonucleotides may usefully and typically have a modification, such as one or more phosphorothioate linkages or 2′-OMe analogues.

In some embodiments, the oligonucleotides are nonidentical and noncomplementary in sequence to any portion of 10 or more contiguous nucleotides of the nucleic acid that encodes the protein aggregant. In other embodiments, the oligonucleotides may bear sequence complementarity or identity, at least in part, to a portion of the nucleic acid sequence that encodes the protein aggregant.

In many embodiments, at least one of the oligonucleotide species in the pharmaceutical composition comprises at least one terminal modification, such as a terminal phosphorothioate linkage or 2′-OMe analogue.

In some embodiments, the composition comprises at least two oligonucleotide species differing in one or more of sequence, length, or composition, often as many as 3, 4, 5, or even as many as 10-50 different oligonucleotide species that differ in any one or more of sequence, length, or composition.

The therapeutic method may be used to treat disorders having a protein aggregation or misassembly etiology, such as Alzheimer's disease, Huntington's disease, cystic fibrosis, amyotrophic lateral sclerosis, Parkinson's disease, spinobulbar muscular atrophy, spinocerebellar ataxia types 1, 2, 3, 6, and 7, dentatorubral-pallidoluysian atrophy, prion diseases, scrapie, bovine spongiform encephalopathy, CJD, new variant CJD, Pick's disease, diabetes type II, multiple myeloma-plasma cell dyscrasias, medullary carcinoma of the thyroid, chronic renal failure, congestive heart failure, chronic inflammation, atherosclerosis (apoA1), and familial amyloidosis.

### BRIEF DESCRIPTION OF THE DRAWINGS

The above and other objects and advantages of the present invention will be apparent upon consideration of the following detailed description taken in conjunction with the accompanying drawings, in which like characters refer to like parts throughout, and in which:

FIG. 1A is a flow chart displaying an experimental protocol for assaying oligonucleotides in cells cultured in vitro for their ability to disrupt or prevent aggregation of a protein aggregant, according to the present invention;

FIG. 1B is a flow chart displaying in greater detail an experimental protocol for assaying oligonucleotides in cells cultured in vitro for their ability to disrupt or prevent aggregation of a protein aggregant, according to the present invention;

FIG. 1C is a flow chart schematizing an experimental protocol for assaying olignucleotides in cells cultured in vitro for their ability to disrupt or prevent aggregation of a protein aggregant, based upon cellular survival, according to the present invention;

FIGS. 2A-2C are fluorescence micrographs of PC12 cell cultures in which aggregates of huntingtin-GFP fusion protein appear light against a dark background, with FIG. 2A showing a control culture, FIG. 2B showing a diminution in aggregate formation after transfection with oligonucleotide Kan uD3T/25G according to the present invention, and FIG. 2C showing a similar degree of diminution of aggregate formation after transfection with oligonucleotide Kan uD12T/25G according to the present invention (micrographs not to same scale);

FIGS. 3A-3C are fluorescence micrographs of PC12 cell cultures in which aggregates of huntingtin-GFP fusion protein appear light against a dark background, with FIG. 3A showing a control culture, FIG. 3B showing a diminution in aggregate formation after transfection with oligonucleotide Kan uRD3/25G according to the present invention, and FIG. 3C showing the effect of transfecting with oligonucleotide Kan uR/25G according to the present invention;

FIGS. 4A-4E show fluorescence micrographs of PC12 cultures, with aggregates of huntingtin-GFP fusion proteins appearing light against a dark background, with FIGS. 4D and 4E showing untransfected control cultures, and FIGS. 4A-4C showing cultures transfected with three different oligonucleotides according to the present invention, as indicated;

FIG. 5 is a chart quantifying aggregate formation in a cell-based assay performed with the indicated oligonucleotides essentially in accordance with the protocol of FIG. 1B, with the visible aggregates scored according to an odds ratio, the average fraction of cells containing aggregates reported in parentheses, and the error bar indicating the standard deviation calculated from averages of four independent experiments;

FIG. 6 is a chart quantifying aggregate formation in a cell-based assay performed with the indicated oligonucleotides essentially in accordance with the protocol of FIG. 1B, with the visible aggregates scored according to an odds ratio, the average fraction of cells containing aggregates reported in parentheses, and the error bar indicating the standard deviation calculated from averages of four independent experiments;

FIG. 7 charts cell survival after induction of mutant huntingtin production in the absence and in the presence of oligonucleotide HDS-9, according to the protocol schematized in FIG. 1C;

FIG. 8 is a photomicrograph demonstrating the ready visualization of live cells attached to the flask surface in the cell survival assay of the present invention; and

FIG. 9A shows recombinant mutant huntingtin N-terminal aggregates captured on a cellulose acetate filter after incubation with the indicated oligonucleotides or compounds, according to the present invention, with FIG. 9B tabulating the quantity of aggregates as a percentage of aggregates observed in the negative control.

### DETAILED DESCRIPTION

The present invention is based upon the discovery that compositions comprising oligonucleotides as short as about 4 nucleotides in length, and as long as about 25 nt in length, can effect the disruption of proteins that are pathologically aggregated within cells (hereinafter also called “protein aggregants” or “aggregants”). The effect can be observed with oligonucleotides that bear no identifiable sequence relationship to the sequence of the gene encoding the protein aggregant. Given the ease with which oligonucleotides can be synthesized, the ease with which they can be delivered to the interior of cells, the lack of systemic toxicity, and the wealth of dosing experience derived from a decade or more of antisense approaches, the use of short oligonucleotides to disrupt protein aggregations provides significant advantages over approaches currently being contemplated to treat these diseases.

The oligonucleotides used in the compositions and methods of the present invention can be as short as 4 nucleotides in length, and as long as 25 nucleotides in length, and thus can be 4, 5, 6, 7, 8, 9, 10, 11, 12, 13, 14, 15, 16, 17, 18, 19, 20, 21, 22, 23, 24, or 25 nucleotides in length, exclusive of optional terminal blocking groups.

The oligonucleotides can comprise nucleobases naturally found in nature in native 5′-3′ phosphodiester internucleoside linkage—e.g., DNA, RNA, or chimeras thereof—or can contain any or all of nucleobases not found in nature (non-native nucleobases), normative internucleobase bonds, or post-synthesis modifications, either throughout the length of the oligonucleotide or localized to one or more portions thereof.

For example, the oligonucleotides of the present invention may usefully comprise altered, often nuclease-resistant, internucleoside bonds, as are typically used in antisense applications. See, e.g., Hartmann et al. (eds.), Manual of Antisense Methodology (Perspectives in Antisense Science), Kluwer Law International (1999) (ISBN:079238539X); Stein et al. (eds.), Applied Antisense Oligonucleotide Technology, Wiley-Liss (cover (1998) (ISBN: 0471172790); Chadwick et al. (eds.), Oligonucleotides as Therapeutic Agents Symposium No. 209, John Wiley & Son Ltd (1997) (ISBN: 0471972797), the disclosures of which are incorporated herein by reference in their entireties.

Modified oligonucleotide backbones may include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, the disclosures of which are incorporated herein by reference in their entireties.

Other modified oligonucleotide backbones useful in the oligonucleotides of the present invention include those that lack a phosphorus atom, such as backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above backbones include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, the disclosures of which are incorporated herein by reference in their entireties.

The oligonucleotides of the present invention may also include normaturally occurring nucleobases, either in standard phosphodiester linkage, where the chemistry allows, or with other types of linkage not found in naturally occurring nucleic acids (as would be clear to the person skilled in the art, various nucleobases which previously have been considered normaturally occurring have subsequently been found in nature).

The oligonucleotides of the present invention may thus include nucleobases such as the known purine and pyrimidine heterocycles, and also heterocyclic analogues and tautomers thereof. Illustrative examples of nucleobases are adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosine, N⁶,N⁶-ethano-2,6-diaminopurine, 5-methylcytosine, 5-(C³-C⁶)-alkynylcytosine, 5-fluorouracil, 5-bromouracil, pseudoisocytosine, 2hydroxy-S-methyl-4-triazolopyridine, isocytosine, isoguanine, inosine and the “non-naturally occurring” nucleobases described in U.S. Pat. No. 5,432,272, included herein by reference in its entirety.

Among non-native nucleobases useful in the oligonucleotides of the present invention, locked nucleic acid (LNA) analogues may have utility for particular protein aggregants, although LNA-containing oligonucleotides tested to date have proven poorly effective in disaggregating huntingtin aggregates, as further described in the Examples below.

LNAs are bicyclic and tricyclic nucleoside and nucleotide analogues and the oligonucleotides that contain such analogues. The basic structural and functional characteristics of LNAs and related analogues are disclosed in various publications and patents, including WO 99/14226, WO 00/56748, WO 00/66604, Wo 98/39352, U.S. Pat. No. 6,043,060, and U.S. Pat. No. 6,268,490, all of which are incorporated herein by reference in their entireties.

The oligonucleotides of the present invention may also usefully include 2′-O-alkyl analogues, such as 2′-OMe analogues; when linked to deoxyribonucleotides in 5′-3′ phosphodiester bonds, the resulting oligonucleotide is a chimera of RNA and DNA.

Differences from nucleic acid compositions found in nature—e.g., altered internucleoside linkages, normaturally occurring nucleobases, and post-synthetic modifications—can be present throughout the length of the oligonucleotide or can instead be localized to discrete portions thereof.

The oligonucleotides useful in the present invention can also optionally include end-groups, at either or both of the 5′ and 3′ termini; such end-groups may usefully reduce degradation or, in addition or in the alternative, provide other functionalities.

For example, the 5′ terminus may be phosphorylated, either chemically or enzymatically, thus increasing the oligonucleotide's negative charge.

The 5′ end may, in the alternative, be modified to include a primary amine group, typically appended during solid phase synthesis through use of an amino modifying phosphoramidite, such as a β-cyanoethyl (CE) phosphoramidite (Glen Research, Inc., Sterling, Va.). The 5′ end may instead be modified to display a reactive thiol group, which can be appended during solid phase synthesis through use of a thiol modified phosphoramidite, such as (S-Trityl-6-mercaptohexyl)-(2-cyanoethyl)-(N,N-diisopropyl)-phosphoramidite (Glen Research, Inc., Sterling, Va.).

Amine and thiol-modified oligonucleotides can be readily conjugated to other moieties, such as proteins, lipids, or carbohydrates.

Among such moieties are usefully those that serve to target the oligonucleotide to the cell type of therapeutic interest.

For example, international patent publications WO 02/47730 and WO 00/37103, incorporated herein by reference in their entireties, describe compounds for intracellular delivery of therapeutic moieties to nerve cells. The targeting moieties are neurotrophins—such as NGF, BDNF, NT-3, NT-4, NT-6, and fragments thereof—that effect the targeted internalization of the compound by nerve cells of various classes. Such moieties may usefully be appended to the oligonucleotides of the present invention that are intended to disrupt protein aggregations characteristic of neurological disorders, such as spinobulbar muscular atrophy, spinocerebellar ataxia types 1, 2, 3, 6, and 7, dentatorubral-pallidoluysian atrophy, Parkinson's disease, and the prion-based encephalopathies.

Other targeting moieties that may usefully be appended to the oligonucleotides of the present invention facilitate passage across the blood brain barrier, such as the OX26 monoclonal antibody (reviewed in Pardridge, “Brain drug delivery and blood-brain barrier transport”, Drug Delivery 3:99-115 (1996), incorporated herein by reference in its entirety; see also U.S. Pat. Nos. 5,154,924 and 5,977,307, incorporated herein by reference in their entireties), or target liver cells, such as lactosaminated albumin (Ponzetto et al., Hepatology 14(1):16-24 (1991), incorporated herein by reference in its entirety).

The 3′ end of the oligonucleotide of the present invention may similarly be amine or thiol modified to permit the ready conjugation of the oligonucleotide to, among others, proteins, carbohydrates, and lipids.

Other 5′ and 3′ end-modifications include, for example, fluorescent labels, that permit the monitoring of the extracellular and intracellular distribution of the oligonucleotide.

Fluorescent labels useful for endmodification are well known, and include, for example, fluorescein isothiocyanate (FITC), allophycocyanin (APC), R-phycoerythrin (PE), peridinin chlorophyll protein (PerCP), Texas Red, Cy3, Cy5, Cy7, and fluorescence resonance energy tandem fluorophores such as PerCP-Cy5.5, PE-Cy5, PE-Cy5.5, PE-Cy7, PE-Texas Red, and APC-Cy7.

Other fluorophores usefully appended to the 5′ or 3′ ends of the oligonucleotides of the present invention include, inter alia, Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3.5, and Cy5.5.

The oligonucleotides may also include a 3′ and/or 5′ group useful for secondary labeling or purification, such as biotin, dinitrophenyl, or digoxigenin.

When the sequence desired for the oligonucleotide is known, the oligonucleotides of the present invention can usefully be synthesized using standard solid phase chemistries appropriate to the nucleobases and linkages desired.

When the sequence desired is yet to be determined, the oligonucleotides of the present invention can usefully be synthesized combinatorially, providing oligonucleotides of all possible sequences for any desired length of oligonucleotide, from which desired sequences can thereafter be selected.

Such combinatorial methods are known in the art. In the simplest such method, all possible nucleobase monomers are used in each synthesis cycle. A disadvantage of this approach is that oligonucleotides of disparate sequence are present in admixture. Other methods permit high throughput parallel synthesis in which oligonucleotides differing in sequence are segregated. See, e.g., Cheng et al., “High throughput parallel synthesis of oligonucleotides with 1536-channel synthesizer,” Nucl. Acids Res. 30(18):e93 (2002).

In one aspect, therefore, the invention provides a method for identifying, from a plurality of oligonucleotides differing in sequence, those oligonucleotides that are effective to disrupt aggregation of a protein aggregant in a cell.

The method comprises introducing each of a plurality of oligonucleotides of disparate sequence separately into cells that have or are likely to develop protein aggregates, and identifying the oligonucleotide that is most effective at disrupting or preventing aggregation.

The oligonucleotides to be tested differ in sequence. They may optionally differ additionally in composition, such as in length, in the presence, position, and number of normative internucleoside linkages, in the presence, position, number and chemistry of normative nucleobases, and in the presence, position, and number of terminal modifications.

The cells are typically cultured cells, and the oligonucleotides are thus introduced into the cells in vitro. In other embodiments, however, the cells are present within a laboratory animal, and the oligonucleotides are introduced by administration to the animal.

The cells chosen for use in this method exhibit or develop aggregation of proteins that are desired to disrupted.

For example, when oligonucleotides are desired to be selected to reduce or prevent aggregation of huntingtin, the cells chosen for use in the assay exhibit or develop huntingtin aggregation; one such cell line is described in Example 1, below.

Similarly, when the oligonucleotides are desired to reduce or prevent aggregation of Aβ (Amyloid plaque of β secondary structure), the cells chosen exhibit or develop Aβ aggregation. When the oligonucleotides are desired to reduce or prevent aggregation of α-synuclein, the cells chosen exhibit or develop α-synuclein aggregation. Other protein aggregants useful in the assays of the present invention include, e.g., atropin-1, ataxin-1, ataxin-2, ataxin-3, ataxin-7, alpha 1A, a tau protein, PrPSc, and transthyretin.

The cells can be naturally occurring, e.g. derived from a patient having the disorder desired to be treated, or can be engineered. Accordingly, the protein aggregation can comprise a naturally-occurring, albeit pathologically aggregated, protein aggregant, or can comprise a non-naturally occurring protein aggregant.

Among non-naturally occurring protein aggregations, fusions that comprise the protein aggregant, or an aggregation-competent portion thereof, and a detectable marker, are particularly useful.

Among such detectable markers, fluorescent proteins having a green fluorescent protein (GFP)-like chromophore prove particularly useful.

As used herein, “GFP-like chromophore” means an intrinsically fluorescent protein moiety comprising an 11-stranded β-barrel (β-can) with a central α-helix, the central α-helix having a conjugated n-resonance system that includes two aromatic ring systems and the bridge between them. By “intrinsically fluorescent” is meant that the GFP-like chromophore is entirely encoded by its amino acid sequence and can fluoresce without requirement for cofactor or substrate. For example, the PC12 neuronal cell lines described in Example 1, below, contain an engineered HD gene exon 1 containing alternating, repeating codons . . . CAA CAG CAG CAA CAG CAA . . . fused to an enhanced GFP (green fluorescent protein) gene.

The GFP-like chromophore can be selected from GFP-like chromophores found in naturally occurring proteins, such as A. victoria GFP (GenBank accession number AAA27721), Renilla reniformis GFP, FP583 (GenBank accession no. AF168419) (DsRed), FP593 (AF272711), FP483 (AF168420), FP484 (AF168424), FP595 (AF246709), FP486 (AF168421), FP538 (AF168423), and FP506 (AF168422), and need include only so much of the native protein as is needed to retain the chromophore's intrinsic fluorescence. Methods for determining the minimal domain required for fluorescence are known in the art. Li et al., “Deletions of the Aequorea victoria Green Fluorescent Protein Define the Minimal Domain Required for Fluorescence,” J. Biol. Chem. 272:28545-28549 (1997).

Alternatively, the GFP-like chromophore can be selected from GFP-like chromophores modified from those found in nature. Typically, such modifications are made to improve recombinant production in heterologous expression systems (with or without change in protein sequence), to alter the excitation and/or emission spectra of the native protein, to facilitate purification, to facilitate or as a consequence of cloning, or are a fortuitous consequence of research investigation.

The methods for engineering such modified GFP-like chromophores and testing them for fluorescence activity, both alone and as part of protein fusions, are well-known in the art. Early results of these efforts are reviewed in Heim et al., Curr. Biol. 6:178-182 (1996), incorporated herein by reference in its entirety; a more recent review, with tabulation of useful mutations, is found in Palm et al., “Spectral Variants of Green Fluorescent Protein,” in Green Fluorescent Proteins, Conn (ed.), Methods Enzymol. vol. 302, pp. 378-394 (1999), incorporated herein by reference in its entirety.

A variety of such modified chromophores are now commercially available and can readily be used in the fusion proteins of the present invention.

For example, EGFP (“enhanced GFP”), Cormack et al., Gene 173:33-38 (1996); U.S. Pat. Nos. 6,090,919 and 5,804,387, is a red-shifted, human codon-optimized variant of GFP that has been engineered for brighter fluorescence, higher expression in mammalian cells, and for an excitation spectrum optimized for use in flow cytometers. EGFP can usefully contribute a GFP-like chromophore to the fusion proteins of the present invention. A variety of EGFP vectors, both plasmid and viral, are available commercially (Clontech Labs, Palo Alto, Calif., USA), including vectors for bacterial expression, vectors for N-terminal protein fusion expression, vectors for expression of C-terminal protein fusions, and for bicistronic expression.

Toward the other end of the emission spectrum, EBFP (“enhanced blue fluorescent protein”) and BFP2 contain four amino acid substitutions that shift the emission from green to blue, enhance the brightness of fluorescence and improve solubility of the protein, Heim et al., Curr. Biol. 6:178-182 (1996); Cormack et al., Gene 173:33-38 (1996). EBFP is optimized for expression in mammalian cells whereas BFP2, which retains the original jellyfish codons, can be expressed in bacteria; as is further discussed below, the host cell of production does not affect the utility of the resulting fusion protein. The GFP-like chromophores from EBFP and BFP2 can usefully be included in the fusion proteins of the present invention, and vectors containing these blue-shifted variants are available from Clontech Labs (Palo Alto, Calif., USA).

Analogously, EYFP (“enhanced yellow fluorescent protein”), also available from Clontech Labs, contains four amino acid substitutions, different from EBFP, Ormo et al., Science 273:1392-1395 (1996), that shift the emission from green to yellowish-green. Citrine, an improved yellow fluorescent protein mutant, is described in Heikal et al., Proc. Natl. Acad. Sci. USA 97:11996-12001 (2000). ECFP (“enhanced cyan fluorescent protein”) (Clontech Labs, Palo Alto, Calif., USA) contains six amino acid substitutions, one of which shifts the emission spectrum from green to cyan. Heim et al., Curr. Biol. 6:178-182 (1996); Miyawaki et al., Nature 388:882-887 (1997). The GFP-like chromophore of each of these GFP variants can usefully be included in fusion protein aggregants of the present invention.

The GFP-like chromophore can also be drawn from other modified GFPs, including those described in U.S. Pat. Nos. 6,124,128; 6,096,865; 6,090,919; 6,066,476; 6,054,321; 6,027,881; 5,968,750; 5,874,304; 5,804,387; 5,777,079; 5,741,668; and 5,625,048, the disclosures of which are incorporated herein by reference in their entireties.

Recombinant fusions of the protein aggregant (or aggregation-competent fragment thereof) with a detectable marker, such as a protein comprising a GFP-like chromophore, makes it possible to detect aggregation, and disruption of aggregation, by qualitative or quantitative observation of the cellular location and local concentration of the protein aggregant.

Where the fused marker is fluorescent, e.g. a protein moiety having a GFP-like chromophore, aggregation can be observed visually, typically using a fluorescence microscope. High throughput apparatus, such as the Amersham Biosciences IN Cell Analysis System and Cellomics® ArrayScan HCS System permit the subcellular location and concentration of fluorescently tagged moieties to be detected and quantified, both statically and kinetically. See also, U.S. Pat. No. 5,989,835, incorporated herein by reference in its entirety.

Markers other than fluorescent markers may be used, and markers need not be fused recombinantly to the aggregating protein.

For example, the protein can usefully be fused recombinantly to a tag that is recognized, and can thus be stained specifically by, an antibody.

Such tags include, for example, a myc tag peptide, the Xpress epitope, detectable by anti-Xpress antibody (Invitrogen Corp., Carlsbad, Calif., USA), the V5 epitope, detectable by anti-V5 antibody (Invitrogen Corp., Carlsbad, Calif., USA), FLAG® epitope, detectable by anti-FLAG® antibody (Stratagene, La Jolla, Calif., USA).

Other useful tags include, e.g., polyhistidine tags to facilitate purification of the recombinant fusion protein aggregant by immobilized metal affinity chromatography, for example using NiNTA resin (Qiagen Inc., Valencia, Calif., USA) or TALON™ resin (cobalt immobilized affinity chromatography medium, Clontech Labs, Palo Alto, Calif., USA); a calmodulin-binding peptide tag, permitting purification by calmodulin affinity resin (Stratagene, La Jolla, Calif., USA), and glutathione-S-transferase, the affinity and specificity of binding to glutathione permitting purification using glutathione affinity resins, such as Glutathione-Superflow Resin (Clontech Laboratories, Palo Alto, Calif., USA), with subsequent elution with free glutathione.

Without intending to be bound by theory, it is possible that oligonucleotides having an inhibitory effect on protein aggregation may be found to associate physically with the misassembled proteins. Isolating the protein aggregant under conditions suitable for continued binding of the oligonucleotide to the protein aggregant may thus permit enrichment for those oligonucleotides that have greatest affinity for the protein aggregant. See Kazantsev et al., Nature Genetics 30:367-76 (2002), incorporated herein by reference in its entirety.

Markers need not be fused recombinantly to the protein aggregant. For example, the protein aggregant can be marked by subsequent staining.

In other embodiments of the method of this aspect of the present invention, the oligonucleotide may be labeled.

Labeling the oligonucleotide is particularly useful for purposes of measuring, and normalizing to, the amount of oligonucleotide that enters the cells being assayed. Labeling of the oligonucleotides also permits the intracellular and extracellular distributions of the oligonucleotides to be assayed.

Typically, when the oligonucleotide is labeled, the protein aggregant is also labeled, since the subcellular distribution of oligonucleotide and protein aggregant may differ and provide complementary information.

The oligonucleotides may, for example, be labeled with a radionuclide, a fluorophore, or a visualizable hapten. When labeled with a radionuclide, the oligonucleotide's subcellular localization may be detected, e.g., using x-ray film or a phosphorimager. When labeled with a fluorophore, the oligonucleotide is typically labeled with a fluorophore having excitation and/or emission spectrum distinguishable from that optionally used to label the protein aggregant, and the oligonucleotide position and concentration is monitored using appropriate fluorescence detection devices.

The oligonucleotides may be labeled during or after synthesis. As described above, the label can be localized to the 5′ and/or 3′ terminus. In addition or in the alternative, the label can be positioned within the oligonucleotide.

When assayed in vitro, the cells used in the methods of this aspect of the invention are typically clonal lines that identically express the protein aggregant. The protein aggregant can be expressed from the cell's chromosome, either from its native locus or from another location into which an engineered construct has been integrated, or from an episomal construct.

When the cells are assayed in culture, the oligonucleotides to be tested for their ability to disrupt protein aggregation can be introduced into the cells by well-known transfection techniques.

Given the short length of the oligonucleotides, the oligonucleotides can be introduced passively, likely by endocytotic mechanisms, without further facilitation.

Alternatively, chemical transfection means can be employed.

For chemical transfection, DNA can be coprecipitated with calcium phosphate or introduced using liposomal and nonliposomal lipid-based agents. Commercial kits are available for calcium phosphate transfection (CalPhos™ Mammalian Transfection Kit, Clontech Laboratories, Palo Alto, Calif., USA), and lipid-mediated transfection can be practiced using commercial reagents, such as LIPOFECTAMINE™ 2000, LIPOFECTAMINE™ Reagent, CELLFECTIN® Reagent, and LIPOFECTIN® Reagent (Invitrogen, Carlsbad, Calif., USA), DOTAP Liposomal Transfection Reagent, FuGENE 6, X-tremeGENE Q2, DOSPER, (Roche Molecular Biochemicals, Indianapolis, Ind. USA), Effectene™, PolyFect®, Superfect® (Qiagen, Inc., Valencia, Calif., USA). Other types of polycations, cationic lipids, liposomes, and polyethylenimine (PEI) are known and may be used.

Mechanical means may also be used, such as electroporation, biolistics, and microinjection. Protocols for electroporating mammalian cells can be found online in Electroprotocols (Bio-Rad, Richmond, Calif., USA) (http://www.bio-rad.com/LifeScience/pdf/New_Gene_Pulser.pdf).

For particle bombardment, see e.g. Cheng et al., Proc. Natl. Acad. Sci. USA 90(10):4455-9 (1993); Yang et al., Proc. Natl. Acad. Sci. USA 87(24):9568-72 (1990).

See also, Norton et al. (eds.), Gene Transfer Methods: Introducing DNA into Living Cells and Organisms, BioTechniques Books, Eaton Publishing Co. (2000) (ISBN 1-881299-34-1), incorporated herein by reference in its entirety.

Each oligonucleotide of distinct sequence and/or composition may be assayed individually, and its effectiveness in disrupting or preventing protein aggregation compared to that of other oligonucleotides. In addition or in the alternative, pools of oligonucleotides may be tested, either to facilitate initial screening or to identify combinations of oligonucleotides with additive or synergistic effect.

In the methods of this aspect of the invention, the oligonucleotides typically will be included within compositions suitable for introduction into cell culture, such as buffered aqueous compositions. Depending upon the duration of the assay, which typically ranges from hours to days, the oligonucleotides may preferably be formulated as sterile aqueous compositions.

Typically, but not invariably, the cells to be tested will be tested in a serum-free medium to prevent adventitious sequestration of the oligonucleotide by proteins in the medium. After introduction of the oligonucleotide into the cells, the degree of protein aggregation is assessed, and the efficacy of the oligonucleotide in disrupting or preventing protein aggregation determined. The efficacy may be measured statically, at any of a variety of time points, or kinetically, and various metrics of efficacy may be used.

For example, the degree of aggregation may measured as the total volume of protein aggregation within the cell at a particular time point after administration; as the number of separately distinguishable aggregates, such as “pinpoint aggregates”; as the greatest density of protein aggregation within the cell at a particular time point after administration; as the difference between greatest and least density of protein aggregation within the cell at a particular time point after administration. For kinetic assays, the effective degree of disruption may be measured as the rate at which the density, or volume, of aggregation dissipates in one or more regions of the cell. The choice among such metrics will be dictated, in part, by the cell type and aggregants selected for assay, and is well within the skill in the art.

The assay method may, and typically will, be repeated, until one or more oligonucleotides, alone or in combination, are identified that possess the desired degree of efficacy.

Other in vitro assays may also be used in this aspect of the invention.

Under some circumstances, protein aggregation can lead to cell death, and oligonucleotides able to inhibit or disrupt aggregation can be identified by their ability to inhibit cell death. See, e.g., Carmichael et al., Proc. Nat'l Acad. Sci. USA, 97:9701-9705 (2000) and Examples 4 and 5, infra.

Although oligonucleotides effective in disrupting or preventing aggregation will typically be chosen through in vitro assays such as those set forth above and in the Examples below, in other embodiments of this aspect of the invention the oligonucleotides will be assayed in vivo using an animal model of protein aggregation. In such in vivo assays, the efficacy of the oligonucleotide can be assessed additionally by using clinical indicia of efficacy, such as diminution or delay of symptoms. In non-human animals, efficacy can also be assessed using post-mortem assays following sacrifice. A variety of such assays are described in the Examples that follow. See also Kazantsev et al., Nature Genetics 30:367-76 (2002), incorporated herein by reference in its entirety.

In a second aspect, the invention provides methods of treating human and animal subjects having disorders of protein assembly. The method comprises administering an effective amount of a composition comprising at least one oligonucleotide species that disrupts or prevents protein aggregation, optionally in admixture with a pharmaceutically acceptable carrier or excipient.

Examples of disorders amenable to treatment in the methods of this aspect of the invention include those set forth in Table 1:

TABLE 1
Amyloidogenic proteins and the amyloid diseases
resulting from their self-assembly into a cross-β
fibril

| Clinical syndrome | Precursor protein | Fibril component |
|---|---|---|
| Alzheimer's | βprotein | β protein 1-40, |
| disease | | 1-41, 1-42, 1-43 |
| Primary systemic | Immunoglobulin | Intact light |
| amyloidosis | light chain | chain or |
| | | fragments thereof |
| Secondary | Serum amyloid A | Amyloid A (76- |
| systemic | | residue fragment) |
| amyloidosis | | |
| Senile systemic | Transthyretin | Transthyretin or |
| amyloidosis | | fragments thereof |
| Familial amyloid | Transthyretin | Over 45 |
| polyneuropathy I | | transthyretin |
| | | variants |
| Hereditary | Cystatin C | Cystatin C minus |
| cerebral amyloid | | 10 residues |
| angiopathy | | |
| Hemodialysis- | β₂-microglobulin | β₂-microglobulin |
| related | | |
| amyloidosis | | |
| Familial amyloid | Apolipoprotein A-1 | Fragments of |
| polyneuropathy | | apolipoprotein A-1 |
| III | | |
| Finnish | Gelsolin | 71 amino acid |
| hereditary | | fragment of |
| systemic | | gelsolin |
| amyloidosis | | |
| Type II diabetes | Islet amyloid | Fragment of IAPP |
| | polypeptide | |
| | (IAPP) | |
| Medullary | Calcitonin | Fragments of |
| carcinoma of the | | calcitonin |
| thyroid | | |
| Spongiform | Prion | Prion or |
| encephalopathies | | fragments thereof |
| Atrial | Atrial | ANF |
| amyloidosis | natriuretic | |
| | factor (ANF) | |
| Hereditary non- | Lysozyme | Lysozyme or |
| neuropathic | | fragments thereof |
| systemic | | |
| amyloidosis | | |
| Injection- | Insulin | Insulin |
| localized | | |
| amyloidosis | | |
| Hereditary renal | Fibrinogen | Fibrinogen |
| amyloidosis | | fragments |

Diseases amenable to treatment by the methods of this aspect of the invention also include, inter alia, huntington's disease, Alzheimer's disease, cystic fibrosis, amyotrophic lateral sclerosis, Parkinson's disease, spinobulbar muscular atrophy, spinocerebellar ataxia types 1, 2, 3, 6, and 7, dentatorubral-pallidoluysian atrophy, prion diseases including scrapie, bovine spongiform encephalopathy, CJD, and new variant CJD.

The administered composition will comprise at least one oligonucleotide prior-demonstrated, either in vitro or in an in vivo model, to disrupt or prevent aggregation of the pathogenic protein, and may include any of the structural modifications described above.

The composition will comprise at least one species of oligonucleotide, and may comprise at least 2, 3, 4, 5, 10, 20, 25, 30, 40 and even as many as 50 to 60 different species, which may differ from one another in any one or more of sequence, length, or composition (such as presence, location, and number of altered internucleobase bonds).

Pharmaceutically acceptable carriers and/or excipients are optionally, but typically, included and are chosen for suitability with the desired method of administration.

Pharmaceutical formulation is a well-established art, and is further described in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X), the disclosures of which are incorporated herein by reference in their entireties, and thus need not be described in detail herein.

Pharmaceutical formulations designed specifically for administration of nucleic acids are also well known.

For example, one exemplary carrier for use with the oligonucleotides of the invention includes nucleic acids, or analogues thereof, that do not themselves possess biological activity per se but that are recognized by in vivo processes that would otherwise reduce the bioavailability of the active oligonucleotides, for example by degrading the active oligonucleotides or promoting their removal from circulation. The coadministration of the active oligonucleotide and carrier nucleic acids, typically with an excess of the inactive material, can result in a substantial reduction of the amount of active nucleic acid recovered in the liver, kidney or other extracirculatory reservoirs, presumably due to competition between the inactive carrier and the nucleic acid for a common receptor. See Miyao et al., Antisense Res. Dev., 5:115-121 (1995); Takakura et al., Antisense & Nucl. Acid Drug Dev., 6:177-183 (1996).

The pharmaceutically acceptable carrier and/or excipient may be liquid or solid and is chosen based, at least in part, upon the desired route of administration so as to provide for the desired bulk, consistency, etc., when combined with the oligonucleotides and the other components of a given pharmaceutical composition.

Routes of administration useful in the practice of this aspect of the invention include both enteral and parenteral routes, including oral, intravenous, intramuscular, subcutaneous, inhalation, topical, sublingual, rectal, intra-arterial, intramedullary, intrathecal, intraventricular, transmucosal, transdermal, intranasal, intraperitoneal, intrapulmonary, and intrauterine.

In treating diseases of protein aggregation or misassembly in neuronal cells, certain routes of administration will require passage of the oligonucleotide active across the blood-brain barrier.

A useful embodiment makes use of neutral liposomes that carry the oligonucleotides and that are decorated on the surface with several thousand strands of polyethyleneglycol (PEG) as described in Pardridge, U.S. Pat. No. 6,372,250. The surface coating prevents the absorption of blood proteins to the surface of the liposome and slows the removal of the liposomes from the blood. It also provides sites for the attachment of ligands recognized by the carrier-mediated transport and receptor-mediated transcytosis systems to allow passage of the liposomes across the blood-brain barrier. In some cases, the ligands mediate the uptake of the pegylated liposomes by cells through the receptor-mediated endocytosis system.

Other methods for treating affected neuronal cells located in the brain utilize an implantable device such as an indwelling catheter through which the oligonucleotides, in an appropriate formulation, can be infused directly onto the neuronal cells. Alternatively, the oligonucleotide formulation is administered intranasally, e.g., by applying a solution containing the oligonucleotides to the nasal mucosa of a patient. This method of administration can be used to facilitate retrograde transport of the oligonucleotides into the brain. The oligonucleotides can thus be delivered to brain cells without subjecting the patient to surgery. See, U.S. Pat. Nos. 5,624,898 and 6,180,603, the disclosures of which are incorporated herein by reference in their entireties.

In another, less preferred, alternative method, the oligonucleotides are delivered to the brain by osmotic shock according to conventional methods for inducing osmotic shock.

Other delivery systems and carriers can be selected that maximize delivery to neuronal cells in the central nervous system, especially in the brain. Such delivery systems and carriers are known to those of skill in the art. These delivery systems include liposomes, foams, wafers, gels and fibrin clots and the like. Delivery systems also include implantable devices such as indwelling catheters and infusion pumps. The delivery method can be selected depending on the location and type of neuronal cells to be treated.

The oligonucleotides of the invention are administered and dosed in accordance with standard medical practice, taking into account the clinical condition of the individual patient, the site and method of administration, scheduling of administration, patient age, sex, body weight and other factors known to medical practitioners.

The pharmaceutically “effective amount” for purposes herein is thus determined by such considerations as are known in the art. The amount is effective either to achieve improvement in clinical signs and/or symptoms—including but not limited to decreased levels of misassembled or aggregated proteins, or improvement or elimination of symptoms and other clinical endpoints—or to delay onset of or progression of signs or symptoms of disease, as are selected as appropriate clinical indicia by those skilled in the art. Cure is not required, nor is it required that improvement or delay, as above described, be achievable in a single dose.

The pharmaceutical composition is preferably administered in an amount effective to reverse protein misassembly and aggregation by at least about 10%, 20%, 30%, 40%, even at least about 50%, 60%, 70%, most preferably at least about 80-100%, although such dramatic effect is not required. It is preferred that the amount administered is an amount effective to maximize reversal of protein misassembly and aggregation while minimizing toxicity.

The dosage can vary depending on the number of cells affected, the location of the cells, the route of administration, the delivery mode, whether treatment is localized or systemic, and whether the treatment is being used in conjunction with other treatment methodologies. Dosages can be determined using standard methodologies. Those skilled in the art can determine appropriate dosages and schedules of administration depending on the situation of the patient.

The composition is preferably administered until reversal of protein misassembly and aggregation is obtained. Preferably the composition is administered from about 2 days up to a year, although chronic lifetime administration is not precluded. Advantageously, the time of administration can be coupled with other treatment methodologies. The oligonucleotide treatment may be applied before, after, or in combination with other treatments such as surgery or treatment with other agents. The length of time of administration can be varied depending on the treatment combination selected.

The present invention will be further understood by reference to the following non-limiting examples.

### EXAMPLE 1

Administration of a Non-Specific Oligonucleotide, Which Does Not Hybridize to the HD Gene, Decreases Aggregate Formation of HD Protein in Cell Culture Studies

PC12 neuronal cell lines, provided by L. Thompson (UCI), are used. See Boado et al., J. Pharmacol. and Experimental Therapeutics 295(1):239-243 (2000), the disclosure of which is hereby incorporated by reference.

Each PC12 cell line has a construct encoding a fusion of HD exon 1 to GFP (see Kazantsev et al., Proc. Nat'l Acad. Sci. USA 96:11404-09 (1999), the disclosure of which is hereby incorporated by reference) integrated into its genome.

These cells thus contain an engineered HD gene exon 1 containing alternating, repeating codons . . . CAA CAG CAG CAA CAG CAA . . . fused to an enhanced GFP gene. Expression of the fusion gene leads to the appearance of green fluorescence co-localized to the site of protein aggregates. The fusion gene is under the control of an inducible promoter regulated by muristerone.

One cell line used in these experiments has a construct with approximately 46 glutamine repeats (encoded by either CAA or CAG); another cell line used in these experiments has about 103 glutamine repeats.

The PC12 cells are grown in DMEM, 5% Horse serum (heat inactivated), 2.5% FBS and 1% Pen-Strep, and maintained in low amounts on Zeocin and G418. 24 hours prior to transfection, the cells are plated in 24-well plates coated with poly-L-lysine coverslips, at a density of 5×10⁵ cells/ml in media without any selection.

Single stranded DNA molecules having no sequence complementarity to the target HD gene are added to the PC12 cells bearing an HD gene exon 1-GFP fusion gene; lacking sequence complementarity, the oligonucleotides do not hybridize appreciably to DNA or RNA encoding Huntingtin protein or its complement. The oligonucleotides are modified as described below.

Transfection conditions are optimized using LipofectAMINE 2000 (“LF2000”) at varying ratios of LF2000 to oligonucleotide.

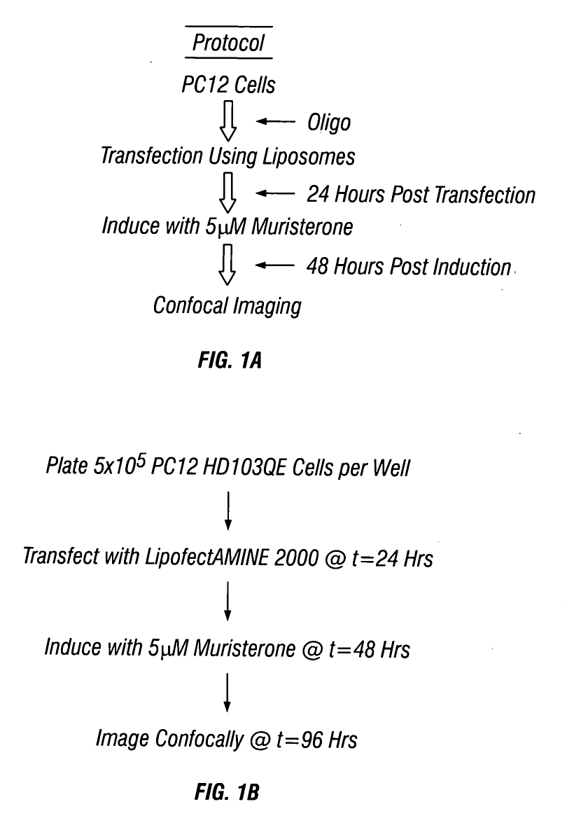

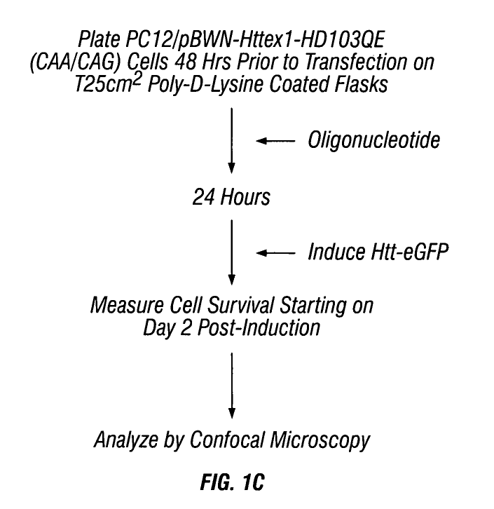

LF2000 is incubated with Opti-Mem I (serum-free medium) for 5 minutes. The oligonucleotide is added and further incubated for 20 minutes at room temperature. The lipid/DNA mixture is applied to the cells and incubated at 37° C. overnight. Muristerone is added after the overnight incubation to induce the expression of HD gene exon 1-GFP. The protocol is schematized in FIG. 1A.

Alternatively, non-specific oligonucleotides are added to the PC12 cells 48 hours after the induction of gene expression by addition of muristerone; and 48-72 hours later, the cells are visualized by confocal microscopy.

Data are acquired on a Zeiss inverted 100M Axioskop equipped with a Zeiss 510 LSM confocal microscope and a Coherent Krypton Argon laser and a Helium Neon laser. Samples are loaded into Lab-Tek II chambered cover glass system for improved imaging. The number of Huntingtin-GFP aggregations within the field of view of the objective is counted in 7 independent experiments. To control for observer bias in counting “pinpoint aggregates”, several scientists are requested to perform an unbiased count of Huntingtin-GFP fusion protein aggregates in various fields from control and treated cell populations.

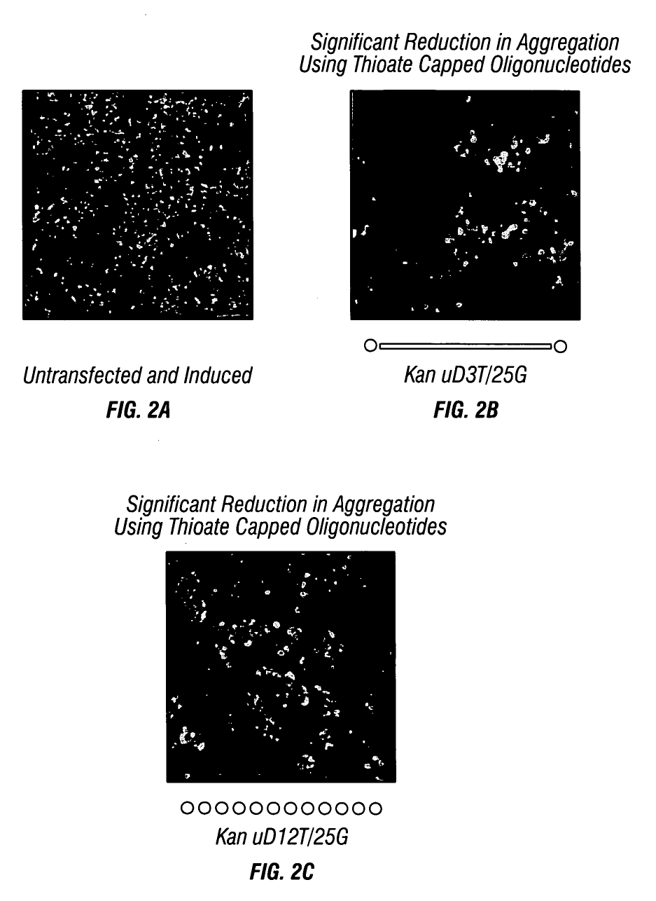

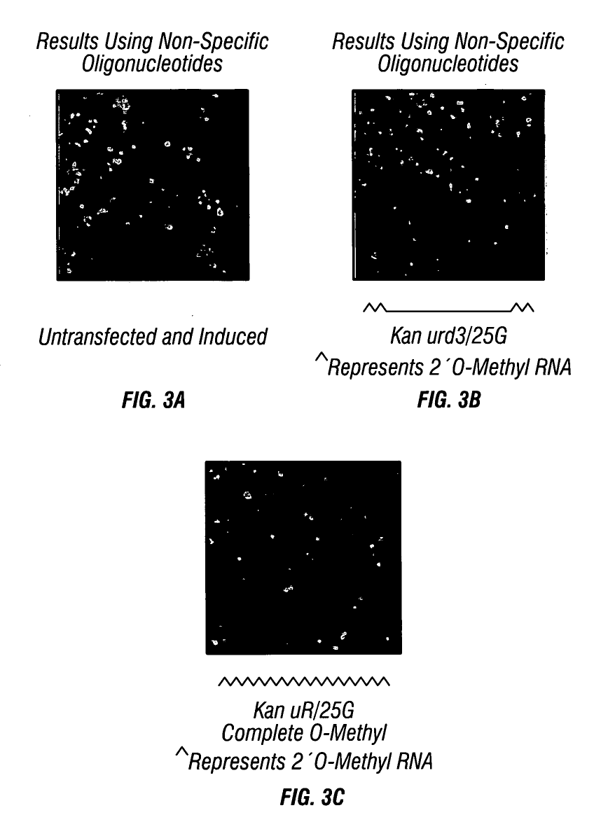

In the absence of oligonucleotide, activation of the promoter leads to high levels of Huntingtin-GFP fusion gene expression and, subsequently, the appearance of Huntingtin-GFP fusion protein aggregates (bright pinpoints), visible in FIGS. 2A and 3A.

A visible reduction in the presence of

Huntingtin-GFP fusion protein aggregates is observed in the presence of an oligonucleotide that does not hybridize appreciably to the HD gene (“non-specific” or “HD non-specific”). The oligonucleotide, denominated “Kan uD3T/25G” has the following sequence and structure:

| | |
|---|---|
| 5′ T*T*G*TGCCCAGTCGTAGCCGAAT*A*G*C 3′ | [SEQ ID NO:1] |
| (Kan uD3T/25G) | |

wherein asterisks indicate phosphorothioate linkages. FIG. 2B shows that administration of Kan uD3T/25G results in a reduction in Huntingtin-GFP fusion protein aggregates, as compared to cells that are induced but not transfected (FIG. 2A).

Similar results are seen with a 25mer HD non-specific single stranded oligonucleotide having all phosphorothioate linkages, denominated Kan uD12T/25G and having the following structure

| | |
|---|---|
| 5′ T*T*G*T*G*C*C*C*A*G*T*C*G*T*A*G*C*C*G*A*A*T*A*G*C 3′ | [SEQ ID NO:2] |
| (Kan uD12T/25G) | |

wherein asterisks indicate phosphorothioate linkages (see FIG. 2C). The degree of reduction is actually similar for both oligonucleotides: FIGS. 2B and 2C are not shot at the same magnification.

Similar results are obtained with Kan uD7T/15G, a 15mer single stranded HD non-specific oligonucleotide with all phosphorothioate linkages, having the following sequence:

| | |
|---|---|
| 5′ G*C*C*C*A*G*T*C*G*T*A*G*C*C*G 3′ | [SEQ ID NO:3] |
| (Kan uD7T/15G) | |

wherein asterisks denote phosphorothioate linkages.

Reduction of Huntingtin-GFP fusion protein aggregate formation is also observed for Kan uRD3/25G:

| | |
|---|---|
| 5′ uugTGCCCAGTCGTAGCCGAATagc 3′ | [SEQ ID NO:4] |
| (Kan uRD3/25G) | |

in which each terminus has three 2′O-Me analogues (shown in lower case). Compare FIG. 3B to FIG. 3A.

However, two other non-specific oligonucleotides (respectively denominated Kan uR/25G and Kan uR/15G) have little to no effect. Kan uR/25G (see FIG. 3C) is a 25-mer containing all 2′-OMe analogues (shown in lower case):

| | |
|---|---|
| 5′ uugugcccagucguagccgaauagc 3′ | [SEQ ID NO:5] |
| (Kan uR/25G) | |

and Kan uR/15G is a 15-mer containing all 2′-OMe analogues (shown in lower case):

| | |
|---|---|
| 5′ gcccagtcgtagccg 3′ | [SEQ ID NO:6] |
| (Kan uR/15G). | |

The reduction in aggregate formation due to the presence of Kan uRD3/25G is not as great as those observed due to the presence of Kan uD3T/25G or Kan uD12T/25G.

In certain experiments, the above-described reduction in Huntingtin-GFP fusion protein aggregate formation effect is observed only when an excess (>25 μg) of an oligonucleotide that is not specific for the HD gene is transfected. The optimal amount of HD non-specific oligonucleotide required to reduce Huntingtin-GFP fusion protein aggregate formation may vary and can be easily determined.

In summary, non-specific single stranded DNA, such as Kan uD3T/25G, which has three phosphorothioates at each terminus, or such as Kan uD12T/25G or Kan uD7T/15G, which are substituted with all phosphorothioates, are effective in reducing the number of HD protein aggregates formed after induction. A single stranded DNA with three 2′-O-methyl RNA at each terminus, such as Kan uRD3/25G, is effective, but less so than Kan uD3T/25G or Kan uD12T/25G. Non-specific double stranded chimeric RNA/DNA oligonucleotides are also less effective in reducing the number of aggregates (not shown). A single stranded oligonucleotide with all 2′-O-methyl RNA residues, such as Kan uR/25G or Kan uR15/G, has little to no effect.

Using this same experimental system, oligonucleotides comprising different lengths, different base composition, different base modifications, and different concentrations are examined to determine optimal length, composition, sequence, and concentration to effect HD disaggregation.

Similar systems are designed to identify oligonucleotides having greatest ability to disaggregate other proteins, such as α-synuclein, Aβ, and prions.

### EXAMPLE 2

Cell Culture Studies Using Short Oligonucleotides of Differing Composition

Further to the experiments of Example 1, several other single stranded DNA molecules are added to PC12 cells bearing an HD gene exon 1-GFP fusion gene. The oligonucleotides include LNA residues, denoted by a “+” prefix, and include the following:

| | |
|---|---|
| 5′ +C + T + CA + GG + AG + T + C + AG + G + TG 3′ | [SEQ ID NO:7] |
| (klo17LNA) | |
| 5′ + T + T + GTGCCCAGTCGTAGCCGAAT + A + G + C 3′ | [SEQ ID NO:8] |
| (Kan klo1:25mer) | |
| 5′ +G + C + C + C + A + G + T + C + G + T + A + G + C + C + G 3′ | [SEQ ID NO:9] |
| (Kan klo2) | |
| 5′ +G + C + C + CAGTCGTA + G + C + C + G 3′ | [SEQ ID NO:1O] |
| (Kan klo3) | |
| 5′ + C + A + G + T + C + G + T +A +G 3′ | [SEQ ID NO:11] |
| (Kan klo4) | |

PC12 cells (Boado et al., J. Pharmacol. and Experimental Therapeutics 295(1): 239-243 (2000)) are used. These particular PC12 cells contain a CAG or CAA repeat of approximately 103 in the CAG/CAA tract, encoding the poly Q tract, in the first exon of the HD gene fused to an eGFP (enhanced GFP) fusion reporter construct. This PC12 cell line has a construct (see Examples 1 and 2 and Kazantsev et al., Proc. Nat'l Acad. Sci. USA 96: 11404-09 (1999)) integrated into its genome. These cells thus contain an engineered HD gene exon 1 containing alternating, repeating codons . . . CAA CAG CAG CAA CAG CAA . . . fused to a GFP gene. As described in Example 1, the HD gene exon 1-GFP fusion gene in these PC12 cells is under the control of an inducible promoter regulated by muristerone.

The protocol described in Example 1 for these PC12 cells (Boado et al., J. Pharmcol Exp Ther. 295(1): 239-243 (2000)) is essentially followed.

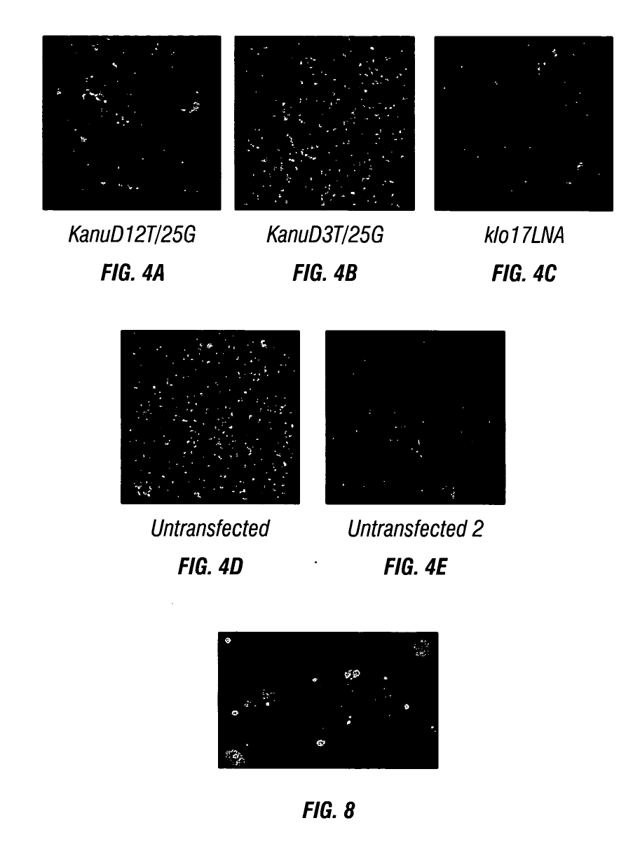

No visible reduction in the appearance of Huntingtin-GFP protein aggregates is observed in the presence of klo17LNA. Indeed, none of the oligonucleotides comprising LNA residues, shown above in this Example, reduces Huntingtin-GFP protein aggregate formation. Kan uD12T/25G has a toxic effect on these cells (i.e., causes more cell death). See FIG. 4A.

### EXAMPLE 3

Cell Culture Studies

In these studies, we test a number of single-stranded DNA oligonucleotides for the capacity to inhibit aggregate formation in PC12 cell lines containing integrated copies of a poly(103)Q-eGFP fusion gene.

PC12 cells (PC12-HD103QE, a gift from Dr. L. Thompson, UCI) are maintained in DMEM, 5% horse serum (heat inactivated), 2.5% FBS, 1% Pen-Strep, 0.2 mg/ml Zeocin, and 100 μg/ml G418. Cells are plated in Lab-Tek II vessels coated with poly-D-lysine coverslips at a density of 5×10⁵ cells for 24 hours prior to transfection in media without selection.

Transfection conditions are found to be best with 2 μg/ml LipofectAMINE 2000 (LF2000, Invitrogen Corp., Carlsbad, Calif.). The samples are incubated with Opti-Mem I reduced-serum medium (Invitrogen Corp., Carlsbad, Calif.) for 5 minutes, followed by the addition of the oligonucleotide; incubation continues for 20 minutes at room temperature. The lipid/oligonucleotide mixture is then applied and, 24 hours later, the cells induced with 5 mM muristerone (Invitrogen Corp.) for fusion gene expression.

Protein aggregates are monitored for 72 hours post-transfection using a Zeiss inverted 100M Axioskop equipped with a Zeiss 510 LSM confocal microscope and a Coherent Krypton Argon laser and a Helium Neon laser.

Approximately 500 cells per field of view are analyzed using a 100× objective in multiple (≧3) randomly chosen fields, and counts are tallied from blinded samples. The percentage of cells containing aggregates is calculated by dividing the number of cells bearing inclusion bodies by the total number of cells expressing the HD-eGFP fusion protein. To be scored positive, a cell must have one or more aggregates. The number of cells with aggregates is determined by incorporating the average of four independent experiments.

The Odds ratio, which is derived by the proportion of eGFP-expressing cells with inclusions divided by proportion of eGFP-expressing cells treated with liposome but no targeting vector, and the standard deviation are determined as described by Carmichael et al., Proc. Natl. Acad. Sci. USA 97:9701-9705 (2000).

Sequences of the oligonucleotides used in this study are shown below. These oligonucleotides variously contain modifications such as phosphorothioate (*) internucleoside linkages, Locked Nucleic Acid (LNA) residues (+), or 2′-O-methyl residues (lower case).

| | |
|---|---|
| HD3S/53T | |
| 5′-C*G*A*GTCCCTCAAGTCCTTCCAACAGCTGCAACAGCAACAACAGCAGCAAC*A*G*A-3′ | [SEQ ID NO:12] |
| HD3S/53 | |
| 5′-G*C*T*GTTGCTGCTGTTGTTGCTGTTGCAGCTGTTGGAAGGACTTGAGGGAC*T*C*G-3′ | [SEQ ID NO:13] |
| HD3S/15 | |
| 5′-C*T*G*TTGCAGCTG*T*T*G-3′ | [SEQ ID NO:14] |
| HD3S/9 | |
| 5′-T*T*G*CAG*C*T*G-3′ | [SEQ ID NO:15] |
| HD1S/9 | |
| 5′-T*TGCAGCT*G-3′ | [SEQ ID NO:16] |
| HD1S/6 | |
| 5′-C*AGCT*G-3′ | [SEQ ID NO:17] |
| HD1S/9-NS | |
| 5′-G*TCGTAGC*C-3′ | [SEQ ID NO:18] |
| HD1S/6-NS | |
| 5′-G*TCGT*A-3′ | [SEQ ID NO:19] |

HD3S/53T is a 53-mer containing three phosphorothioate linkages at each terminus, and is complementary to the transcribed strand of the HD gene. HD3S/53 is identical in structure, but is complementary to the nontranscribed (NT) strand. Similarly, HD3S/15 and HD3S/9 are complementary to the NT strand, but are 15 and 9 bases in length, respectively. HD1S/9 and HD1S/6 are also “specific NT” oligos—that is, complementary to the HD gene—but contain only one phosphorothioate linkage on each terminus. The shorter sequences encompass the central region of the HD-sequence contained in the 53-mer. In other oligonucleotides, the number of phosphorothioate linkages are reduced in the 9-mers and 6-mers from three to one in order to maintain the approximate ratio between the number of thioate and diester bonds linking oligomers differing in length (53 to 15 to 9 to 6).

Others of the oligonucleotides, HD1S/9-NS and HD1S/6-NS, are nonspecific (NS) oligomers (i.e., bear no significant complementarity to the HD-gene sequence), and contain a single phosphorothioate linkage on each end.

Each of the oligonucleotides is transfected at the same molar amount into a PC12 rat pheochromocytoma cell line containing an unknown number of integrated copies of the fusion gene HD103QE. This integrated gene is a truncated Htt sequence containing 103 CAG repeats, fused at the C terminus to an enhanced green fluorescent protein (eGFP) tag, and inducible by muristerone (Kazantsev et al., Proc. Natl. Acad. Sci. USA 96:11404-11409 (1999)). A molar amount equivalent to 5 μg for each oligonucleotide does not exhibit cell toxicity, whereas 25μg equivalents of each oligonucleotide lead to variable results and, in some cases, persistent toxicity.

FIG. 1B schematizes the experimental protocol used to evaluate the impact of these oligomers on the inhibition of aggregate formation. To initialize the reaction, cells are maintained in low amounts of Zeocin and G418 and plated in Lab-Tek II chambers coated with poly-D-lysine coverslips. The oligos are transfected using LF2000 and, 24 hours later, the cells are induced with muristerone. Protein aggregates initially appear after 24 hours and reached a maximum between 48 and 72 hours post-induction.

Samples are loaded into a Lab-Tek II chambered cover-glass element to improve image analyses and enable quantification. Inhibition of protein aggregation is measured by viewing cells in a Zeiss inverted 100M Axioskop confocal microscope (510LSM) using a Coherent Krypton Argon and Helium Neon laser. Inclusions are counted blindly in three randomly-selected fields of view, each containing approximately 500 cells.

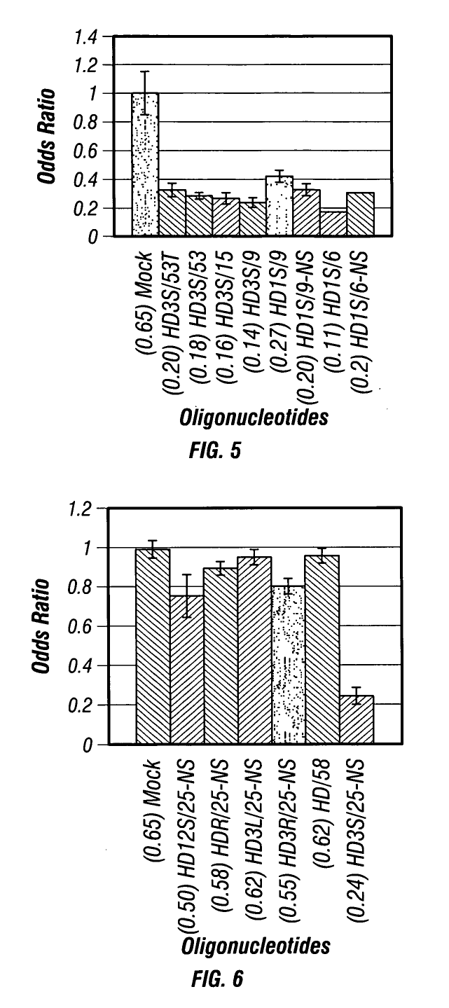

Results from the average of four independent experiments are shown in FIG. 5.

The number of aggregates or inclusions is expressed as an odds ratio, derived by dividing the proportion of eGFP-expressing cells containing aggregates by the proportion of eGFP-expressing cells in a mock transfection with liposomes but with no added oligonucleotide. This is an appropriate statistical value for scoring visible aggregates because the total number of cells expressing eGFP can vary from day to day, but the relative proportion of cells with inclusions varies only slightly from experiment to experiment. Since the cell line used in our experiments is a single clonal isolate, the percentage of cells expressing eGFP is over 90%. Cell viability is found to be consistent among experiments (>90% via Trypan Blue staining).

FIG. 5 represents data obtained from experiments analyzed 72 hours after transfection, a time-point that enables sufficient expression of the fusion gene and adequate time for accumulation of the inclusion bodies.

Approximately 90% of the mock transfected cells are seen to express the eGFP fusion protein and, of these cells, 60-65% contain discernible and visible inclusions, often several per cell. In the controls, the induced PC12 cells undergo a mock transfection with the liposome carrier and produce a baseline odds ratio. The number in parenthesis represents the average fraction of cells with aggregates found per field of 500 cells from four separate experiments, while the standard deviation bars represent variance in the odds ratio data.

When certain oligonucleotides are mixed with the liposome and transfected, an inhibition of inclusion formation is observed: oligonucleotides HD3S/53T and HD3S/53 reduce the number of inclusions over 50% even though these oligonucleotides are designed to hybridize to either the transcribed (T) or nontranscribed strand of the HD gene. Shortening the oligonucleotides preserves this property: HD3S/15 (15mer) and HD3S/9 (9-mer) are as effective as the 53mers.

We reduce the number of phosphorothioate linkages proportionally in the 9-mer and the 6-mer so that the ratio of phosphorothioate to phosphodiester base linkages is kept approximately equal to the ratio of modifications in the larger oligomers. As shown in FIG. 5, both the 9-mer and the 6-mer containing a single phosphorothioate linkage at the 3′ and 5′ ends remain active in the inhibition of aggregate formation in the PC12 cells. Elimination of this modification results in nuclease digestion of the oligonucleotide leading to irreproducible and inconsistent results (data not shown).

The half-life of an unmodified oligomer in vivo is often measured in minutes (20), and even providing the cell with a potentially digestible DNA molecule can affect cellular metabolism since the levels of free deoxyribonucleotides in the cell itself are strictly regulated.

Next, we scramble the sequences of the shortest oligomers, so that they have no sequence complementarity to the HD gene. As shown in FIG. 5, HD1S/9-NS and HD1S/6-NS (both non-specific) are also effective in preventing inclusion formation; in fact, the levels of inclusion reduction rival those seen with the HD3S/53 oligomers. Clearly, nucleotide sequence is not a factor in the inhibition of inclusion formation.

Since the length of the oligonucleotide appears not to be an important factor in suppression of inclusion formation, at least within the rage of 53 to 6 nt, we synthesize a series of 25-mers—an average length of the molecular species outlined above—with different base modifications.

The sequences are shown below, with phosphorothioate linkages shown with asterisks and 2′O-Me residues shown in lower case:

| | |
|---|---|
| HD12S/25-NS | |
| 5′-T*T*G*T*G *C*C*C*A*G*T*C*G*T*A*G*C*C*G*A*A*T*A*G*C-3′ | [SEQ ID NO: 20] |
| HD3S/25-NS | |
| 5′-T*T*G*TGCC CAGTCGTAGCCGAAT*A*G*C-3′ | [SEQ ID NO:21] |
| HDR/25-NS | |
| 5′-ttgtgcccagtcgtagccgaatagc-3′ | [SEQ ID NO:22] |
| HD3R/25-NS | |
| 5′-ttgTGCCCAGTCGTAGCCGAATagc-3′ | [SEQ ID NO:23] |
| HD/58 | |
| 5′-CAGCAGCAGTAGCAGCAGCAGCUUUUgcugcugcugcugcugcugcugcuguuuuCAG-3′ | [SEQ ID NO:243] |

HD12S/25-NS is a 25-mer, nonspecific for HD fusion gene, containing all PS linkages.

HD3S/25-NS is a 25-mer, nonspecific for HD fusion gene, containing 3 terminal PS linkages.

HDR/25-NS is a 25-mer, nonspecific for the HD fusion gene, containing all 2′-O-methyl RNA.

HD3R/25-NS is a 25-mer, nonspecific for the HD fusion gene, containing 3 terminal 2′-O-methyl RNA residues.

HD3L/25-NS is a 25-mer, nonspecific for the HD fusion gene, containing 3 LNA residues on each end.

HD/58 is a 58-mer, double-stranded hairpin molecule, specific for the CAG repeat.

As shown in FIG. 6, HD12S/25-NS—a nonspecific oligo with each linkage being phosphorothioate rather than phosphodiester—has an effect on inclusion reduction that is marginal in comparison to those data presented in FIG. 5. The same is true for HDR/25-NS, an oligomer containing all 2′-0methyl RNA bases, a modification that confers nuclease resistance, and for HDL/15-NS, a 15-mer composed of all locked nucleic acids, a modification that alters the structure of the DNA residue and makes it more RNA-like. The importance of terminal phosphorothioate linkages is emphasized by the results obtained with HD3L/25-NS and HD3R/25-NS, two 25-mers with either three locked nucleic acid residues or three 2′-O-methyl RNA residues on each end. Little or no reduction in inclusions is observed when these oligomers are used.

To investigate the importance of the single-strand character of the oligonucleotide, we construct a single-stranded 58-mer that has internal complementarity among 30 bases so that it spontaneously folds into a double-stranded hairpin with no free ends. When this molecule is tested in the PC12 system, it proves ineffective in suppressing aggregation (FIG. 6). As a positive control, we test HD3S/25-NS, a nonspecific 25-mer again with three phosphorothioate linkages on both ends; this molecule is observed to be quite effective in preventing inclusion formation at levels similar to its 53-mer, 15-mer, and 9-mer relatives.

Thus, while the length and sequence of the oligonucleotide appears not to be critical, the number and type of base or linkage modification and the single-strand character appear to be important in the inhibition of inclusion formation in inducible PC12 cells.

### EXAMPLE 4

A Cell Survival Assay For Detecting Disaggregation Of Huntingtin Aggregates

A cell line, PC12/pBWN:httex, containing the first exon of Huntingtin including the 103 polyglutamine repeats (each Q is encoded by either CAA or CAG; essentially alternating CAACAG), fused to the eGFP (enhanced GFP) gene (gift of Dr. Erik Schweitzer, UCLA) is used. This cell line has incorporated a construct with essentially alternating CAACAG encoding for the poly Q tract (see Schweitzer et al., J. Cell. Science 96: 375-381 (1990), the disclosure of which is incorporated by reference herein). The promoter directing expression of the Huntingtin-eGFP fusion is regulated by ecdysone analogues.

These cells are useful because after induction, Huntingtin aggregate formation is overwhelming; eventually, the cells die. Hence, a disruption in Huntingtin aggregate formation ultimately prolongs cell life and proliferation as indicated by sustained green fluorescence. Careful measurements of extending cell life are made.

1×10⁵ cells are passaged in poly-D-lysine coated T25 flasks 4-5 days prior to transfection, as the cells have a slow growth rate. The cells are transfected by using 10 μg Lipofectamine2000 with 5 μg single stranded oligonucleotide mixed with 500 μl Optimem. The following oligonucleotides are tested in this assay: Kan uD3T/25G, Kan uD12T/25G, Kan uRD3/25G.

The cells are induced 24 hours after transfection by the addition of 0.1 μM tebufenozide (day 1). Confocal microscopy photos are taken on days 2, 3, 6 and 7 post-induction.

On day 7 post-induction, there are about 1% cells surviving in flasks treated with oligonucleotide; in contrast, by day 6 post-induction, untreated cells (ut) do not survive.

Thus, treating these cells with single stranded DNA molecules causes disaggregation of the Huntingtin aggregates and increases survival of these cells.

Accordingly, single stranded DNA molecules, non-specific for the HD gene, cause disaggregation of Huntingtin protein aggregates in these cells, which is manifested in these cells as cell survival.

This cell system can be used to identify oligonucleotides that effect disruption of protein aggregation, extending cell survival.

### EXAMPLE 5

Cell Survival Assay Experiments

PC12 cells/pBWN-Httex1-HD103QE (a gift from Dr. E. Schweitzer— UCLA) are maintained in DMEM (high glucose), 5% FBS, 10% horse serum (Invitrogen Corp., Carlsbad, Calif.), 1% Pen-Strep and 25 mM HEPES, pH 7.4 and grown at 37° C. in 9.5% CO₂. Cells (10⁵) are plated 48 hours prior to transfection in poly-D-lysine coated T25 cm² flasks. Transfection is carried out in Opti-Mem I using a ratio of 2:1 of LipofectAMINE 2000 to oligonucleotide.

Following transfection, cells are induced with 0.1 μM tebufenozide for expression of the htt-eGFP fusion. Cell survival measurements are started 24 hours post-induction, using a Zeiss inverted 100M Axioskop equipped with a Zeiss 510 LSM confocal microscope and a Coherent Krypton Argon laser and a Helium Neon laser. Confocal pictures are taken over a period of seven days following induction, and surviving cells are distinguished from nonviable cells by the adherence to the flasks. Approximately 500 cells per field of view are counted in at least five randomly chosen fields and averaged over four independent experiments.

Because inclusions are correlated with HD-related neurotoxicity, we test the effect of oligonucleotides on cell survival with the protocol shown in FIG. 1C. For this, we use an independently derived PC12 line containing HD103QE that, in contrast to the line used in Examples 1-3, dies rapidly after induction (Schweitzer et al., J. Cell Sci. 96:375-381 (1990); Suhr et al., Proc. Natl. Acad. Sci. USA 95:7999-8004 (1998)).

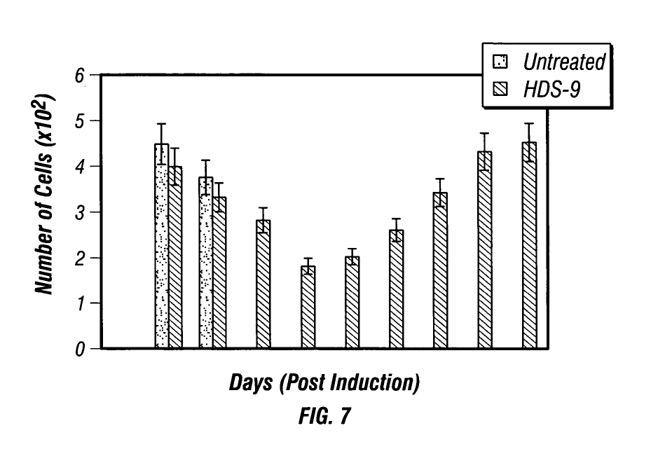

Cells are plated 48 hours prior to transfection with HD1S/9 (9mer)(sequence given in Examples above), which is similar in structure to its related molecule HD3S/9 (sequence given in Examples above) in that it has 1 phosphorothioate linkage at each end. The number of cells surviving 14 days after induction is evaluated by confocal microscopy using the same method as described above. Again, approximately 500 cells per field of view are counted blindly after two days and, on subsequent days, over five different randomly-selected areas in four separate experiments. Measurements of live cells begin 24 hours post induction and subsequent measurements are taken at the time points indicated in FIG. 7.

Cells that undergo the mock transfection die within 72 hours post induction. At day four, no viable cells are observed in the mock-treated samples, but survival is observed through seven days in cells treated with HD1S/9-NS. Dead cells are evident in all of the samples, but those surviving are easily detected by their attachment to the flask surface (FIG. 8).

Nonviable cells detach, round up, and remain suspended in the culture flask; they are easily removed by aspirating the media from the dish prior to counting. After seven days, almost 40% of the cells remain viable, and after 14 days the surviving cell population has more than doubled.

The cell line PC12/pBWN-httex1-HD25QE, which is identical to the PC12 cell line derivative bearing Q103 except that it contains a polyglutamine repeat length of 25, is used as a control to the cell survival assay. Upon induction, we observe only a 5% reduction in cell survival after 7 days of culture and the addition of any oligonucleotides was seen to have no effect on the survival of these cells (data not shown).

### EXAMPLE 6

In Vitro Assays for Inhibition of Aggregation

Several oligonucleotides, including two randomly selected PCR primers, are tested for their inhibitory effect in an in vitro mutant huntingtin aggregation assay.

The assay is performed essentially as described in Huang et al., Somat. Cell Mol. Genet. 24(4):217-33 (1998), the disclosure of which is incorporated herein by reference in its entirety. Briefly, mutant huntingtin N-terminal fragment containing 58 glutamine residues is expressed in bacteria as a GST fusion. The purified fusion protein is treated with protease to release the N-terminal htt fragment, which forms aggregates completely within 24 hours. Experimental samples include 40 μM of oligonucleotides. After aggregate formation is complete, the aggregates are captured on a cellulose acetate membrane with suitable pore size and quantified.

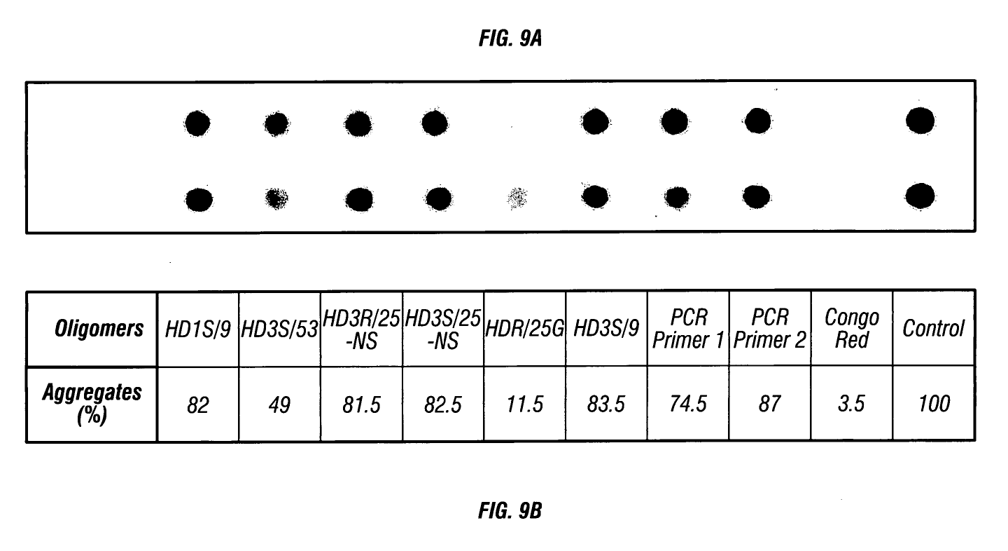

FIGS. 9A and 9B show the effects of the tested oligonucleotides on the formation of huntingtin aggregates.

As expected, a known inhibitor, Congo Red, completely blocks aggregate formation.

All of the oligonucleotides tested showed the inhibitory effect on aggregation, including two random selected PCR primers. This is consistent with results discussed in the Examples above that the inhibition is not sequence specific. Among the tested oligonucleotides, HD3S/53 showed more than 50% inhibition (normalized to negative control) and HDR/25G showed even stronger effect, with more than 85% inhibition.

HDR/25G is identical to HDR/25-NS.

The results suggest a direct interaction between the oligonucleotides and the polyQ tract of mutant huntingtin.

First, longer oligonucleotides showed stronger inhibitory effects than shorter oligonucleotides.

The relationship of length to effectiveness is more easily and directly assessed in this in vitro assay than in the cell-based assays of the earlier Examples, due to the absence of nucleases present within the cultured cells of the cell-based assays. In the in vitro assay used in this Example, oligonucleotides consisting of 9 to 18 nucleotides show the same weak effect. PCR primer 1, a 36mer, shows a stronger effect (25% inhibition) and HD3S/53, a 53mer, shows the strongest inhibitory effect. It is clear that with the increase in length, the inhibitory effect increases.

Second, RNA is much more effective than DNA. At the pH of the in vitro assay, approximately pH 8.0, the additional ribose hydroxyl groups are readily available to form hydrogen bonds. The greater effectiveness of such RNA oligonucleotides suggests that the additional hydrogen bonds are directly involved in the inhibition of aggregates, likely through the formation of hydrogen bond between the oligonucleotide and glutamine residues in the poly-Q tract, with this interaction preventing interaction among huntingtin fragments themselves, in turn preventing the formation of aggregates.

### EXAMPLE 7

Identification of Four Base Single Stranded Oligonucleotide Effective in Disrupting Huntingtin Aggregates

In order to screen a large number of oligonucleotides for the ability to disrupt huntingtin aggregates, all 256 possible four base single stranded oligonucleotides are synthesized.

One set is modified by phosphorothioate linkage of the 5′-most base; another set is modified by phosphorothioate linkage of the 3′-most base; a further set is modified by phosphorothioate linkage throughout the oligonucleotide. All bases are otherwise standard deoxyribonucleotides.

These 4-mer oligonucleotides are tested individually, across a range of concentrations, for their ability to cause disaggregation of Huntingtin protein aggregates according in one or both of the assays set forth respectively in Examples 1-2 and Example 3, and the most effective 4-mer oligonucleotides are identified.

### EXAMPLE 8

Administration of Single Stranded Oligonucleotides to a Transgenic Animal Model System of HD Causes a Reduction of Huntingtin Protein Aggregates

An animal model system for Huntington's disease is obtained.

See, e.g., Brouillet, Functional Neurology 15(4): 239-251 (2000), the disclosure of which is hereby incorporated by reference. See also Ona et al., Nature 399:263-267 (1999), Bates et al., Hum. Mol. Genet. 6(10):1633-7 (1997) and Hansson et al., J. Neurochem. 78:694-703, the disclosure of each of which is hereby incorporated by reference. See also Rubinsztein, Trends in Genetics, 18(4):202-209 (2002), the disclosure of which is hereby incorporated by reference in its entirety.

For example, a transgenic mouse expressing human Huntingtin protein, a portion thereof, or fusion protein comprising human Huntingtin protein, or a portion thereof, with, for example, at least 36 CAG repeats (alternatively, any number of the CAG repeats may be CAA) in the CAG repeat segment of exon 1 encoding the poly-Q tract.

An example of such a transgenic mouse strain is the R6/2 line (Mangiarini et al., Cell 87: 493-506 (1996), the disclosure of which is hereby incorporated by reference in its entirety). The R6/2 mice are transgenic Huntington's disease mice, which overexpress exon one of the human HD gene (under the control of the endogenous promoter). The exon 1 of the R6/2 human HD gene has an expanded CAG/polyglutamine repeat lengths (150 CAG repeats on average). These mice develop a progressive, ultimately fatal neurological disease with many features of human Huntington's disease. Abnormal aggregates, constituted in part by the N-terminal part of Huntingtin (encoded by HD exon 1), are observed in R6/2 mice, both in the cytoplasms and nuclei of cells (Davies et al., Cell 90: 537-548 (1997), the disclosure of which is hereby incorporated by reference). Preferably, the human Huntingtin protein in the transgenic animal has at least 55 CAG repeats and more preferably about 150 CAG repeats. These transgenic animals develop a Huntington's disease-like phenotype.

These transgenic mice are characterized by reduced weight gain and lifespan and motor impairment characterized by abnormal gait, resting tremor, hindlimb clasping and hyperactivity from 8 to 10 weeks after birth (for example the R6/2 strain; see Mangiarini et al., Cell 87: 493-506 (1996)). The phenotype worsens progressively toward hypokinesia. The brains of these transgenic mice also demonstrate neurochemical and histological abnormalities, such as changes in neurotransmitter receptors (glutamate, dopaminergic), decreased concentration of N-acetylaspartate (a marker of neuronal integrity) and reduced striatum and brain size. In addition, abnormal aggregates containing the transgenic part of or full-length human Huntingtin protein are present in the brain tissue of these animals. The R6/2 strain is an example of such a transgenic mouse strain. See Mangiarini et al., Cell 87: 493-506 (1996), Davies et al., Cell 90: 537-548 (1997), Brouillet, Functional Neurology 15(4): 239-251 (2000) and Cha et al., Proc. Nat'l Acad. Sci. USA 95: 6480-6485 (1998).

To test the aggregation-disrupting effect of oligonucleotides in an animal model, different concentrations of Kan uD3T/25G, Kan uD12T/25G, or any of the 4-mer oligonucleotides identified in Example 4, are administered to the transgenic animal, for example by injecting pharmaceutical compositions comprising the oligonucleotides directly into brain ventricles. The progression of the Huntington's disease-like symptoms, for example as described above for the mouse model, is then monitored to determine whether treatment with the oligonucleotides results in reduction or delay of symptoms.

The animal is then sacrificed and brain slices are obtained. The brain slices are then analyzed for the presence of aggregates containing the transgenic human Huntingtin protein, a portion thereof, or fusion protein comprising human Huntingtin protein, or a portion thereof. This analysis includes, for example, staining the slices of brain tissue with anti-Huntingtin antibody and adding a secondary antibody conjugated with FITC which recognizes the anti-Huntingtin's antibody (for example, the anti-Huntingtin antibody is mouse anti-human antibody and the secondary antibody is specific for human antibody) and visualizing the protein aggregates by fluorescent microscopy. Alternatively, the anti-Huntingtin antibody can be directly conjugated with FITC. The levels of Huntingtin's protein aggregates are then visualized by fluorescent microscopy.

### EXAMPLE 9

Administration Of Single Stranded Oligonucleotides To A Transgenic Animal Model System Of HD Increases Longevity

Sixty (60) R6/2 mice (Jackson Laboratories Strain B6CBA-TgN(HDexon1)₆₂ Gpb/J) are used in this study for each oligonucleotide tested.

Mice are acclimated for 7 days and given LabDiet 5K52 and tap water ad libidum. Animals are examined prior to initiation of the study to assure adequate health and suitability; animals that are found to be diseased or unsuitable are not be assigned to the study.

After aclimatization, spontaneously breathing six-week-old R6/2 mice with body weight of approximately 20 g are randomly assigned to one of three treatment groups: mice 1-20 serve as a control for the surgical procedure, mice 21-40 serve as a drug control, and mice 41-60 constitute the experimental group.

At day zero, mice 21-60 are anesthetized and a 2 cm sagittal incision made over the skull. A small opening is made on the cranium 1 mm right lateral to the bregma. A microcannula is inserted to a depth of 3 mm, secured with a dental acrylic, and attached to an Alzet 2004 osmotic pump (Alza Corp., Mountain View, Calif.).

The osmotic pump is filled with 200 μl of vehicle (animals 21-40) or oligonucleotide solution (animals 41-60). The pumps continuously deliver the drug or vehicle for four weeks. The experimenters are blinded to the identity of the drug used in the pump until the death of all the mice in the study.

Dosing continues automatically and continuously for 4 weeks (28 days).

Mice are evaluated daily for survival.

Once every week for the period of the study, each animal is weighed in order to assess possible differences in animal weight among treatment groups as an indication of response to therapy. Animals exhibiting weight loss greater than 20% are euthanized.

Once each week, each animal is tested on the rotarod apparatus. Mice are placed on the rotarod at both 5 and 15 rpm and the test is terminated either when the mouse falls from the rod or at the end of 10 minutes.

Survival and disease progression are determined at the end of the study. Statistical differences between treatment groups are determined using Student's t-test, Wilcoxon matched pair test, Logrank evaluation of survival curves. Body weights are also evaluated for differences between the treatment groups.

Additional details on the experimental procedures are found in Ona et al., Nature 399:263-267 (1999), and Chen et al., Nature Med. 6(7):797801(2000), the disclosures of which are incorporated herein by reference in their entireties.

The following oligonucleotides are tested: HD3S/53T; HD3S/53; HD3S/15; HD3S/9; HD1S/9; HD1S/9-NS; HD1S/6; HD1S/6-NS; HD12S/25-NS; HDR/25-NS; HD3L/25-NS; HD3R/25-NS; HD/58 and HD3S/25-NS.

Survival of mice 41-60 is significantly increased, and their disease progression as assessed by motor performance significantly delayed, as compared to control animals 1-40, in mice treated with HD3S/53T; HD3S/53; HD3S/15; HD3S/9; HD1S/9; HD1S/9-NS; HD1S/6; HD1S/6-NS; and HD3S/25-NS.

### EXAMPLE 10

Administration of Single Stranded Oligonucleotides to a Drosophila Model System of HD Causes a Reduction of Huntingtin Protein Aggregates

A Drosophila melanogaster model system for Huntington's disease is obtained. See, e.g., Steffan et al., Nature, 413: 739-743 (2001) and Marsh et al., Human Molecular Genetics 9: 13-25 (2000), the disclosure of each of which is hereby incorporated by reference. For example, a transgenic Drosophila expressing human Huntingtin protein, a portion thereof (such as exon 1), or fusion protein comprising human Huntingtin protein, or a portion thereof, with, for example, at least 36 CAG repeats (preferably 51 repeats or more) (alternatively, any number of the CAG repeats may be CAA) in the CAG repeat segment of exon 1 encoding the poly Q tract. These transgenic flies are engineered to express human Huntingtin protein, a portion thereof (such as exon 1), or fusion protein comprising human Huntingtin protein, or a portion thereof, in neurons.

To test the effect of the oligonucleotides described in the application in this Drosophila model, different concentrations of Kan uD3T/25G, Kan uD12T/25G, or any of the 4-mer oligonucleotides identified in Example 4, are administered to the transgenic Drosophila, for example by injecting pharmaceutical compositions comprising the oligonucleotides into the brain, by orally administering the oligonucleotides, or by administering the oligonucleotides as part of food. Administration of the oligonucleotides is performed at various stages of the Drosophila life cycle.

The progression of the Huntington's disease-like symptoms is monitored to determine whether treatment with the oligonucleotides results in reduction or delay of symptoms.

One or another of the following assays is additionally performed.

In a first additional assay, disaggregation of the Huntingtin protein aggregates, or reduction in the formation of the Huntingtin protein aggregates, in these flies is monitored.

In a second additional assay, lethality and/or degeneration of photoreceptor neurons are monitored.

Neurodegeneration due to expression of human Huntingtin protein, a portion thereof (such as exon 1), or fusion protein comprising human Huntingtin protein, or a portion thereof, is readily observed in the fly compound eye, which is composed of a regular trapezoidal arrangement of seven visible rhabdomeres (subcellular light-gathering structures) produced by the photoreceptor neurons of each Drosophila ommatidium. Expression of human Huntingtin protein, a portion thereof (such as exon 1), or fusion protein comprising human Huntingtin protein, or a portion thereof, leads to a progressive loss of rhabdomeres.

Results of administration of the oligonucleotides described in the application in this Drosophila model are evaluated and the most effective oligonucleotides identified.

### EXAMPLE 11

Administration of Single Stranded Oligonucleotides to an In vitro Model System of HD Causes a Reduction of Huntingtin Protein Aggregates

A microtiter plate assay for polyglutamine aggregate is obtained. See Berthelier et al., Anal. Biochem. 295:227-236 (2001), the disclosure of which is hereby incorporated by reference.

Following Berthelier et al., poly Q peptides of varying lengths are synthesized. Preferably, these peptides have pairs of Lys residues flanking the poly Q. The peptides can be biotinylated. The peptides can be about Q28. An exemplary peptide is biotinylated K₂Q₃₀K₂. The peptides could be purified.

The peptides are solubilized and disaggregated by essentially the methods described in Berthelier et al., Analytical Biochemistry 295: 227-236 (2001), incorporated herein by reference in its entirety. Poly Q aggregates are then formed from the solubilized peptides as described in Berthelier et al., Analytical Biochemistry 295: 227-236 (2001). The aggregates are collected by centrifugation, resuspended in a buffer (such as PBS, 0.01% Tween 20 and 0.05% NaN₃) and aliquoted into Eppendorf tubes. The tubes are snap-frozen in liquid nitrogen and stored at −80° C. Biotinylated peptides and aggregates of them are prepared essentially as described in Berthelier et al., Analytical Biochemistry 295: 227-236 (2001). 96-well microtiter plates with the aggregates in some or all the wells are prepared essentially as described in Berthelier et al., Analytical Biochemistry 295: 227-236 (2001). In some experiments, 20 ng per well of aggregates are used. Aggregate extension assays are done essentially as described in Berthelier et al., Analytical Biochemistry 295: 227-236 (2001).

The microtiter aggregate extension assay is used to test the ability of the oligonucleotides described in the application, including in the Examples (the oligonucleotides could be different concentrations of HDA3T/53), to inhibit poly Q aggregate extension in this microtiter in vitro aggregate extension assay.

### EXAMPLE 12

Use of a Yeast System to Measure Inhibition of Protein Misassembly by Single Stranded Oligonucleotides

Inhibition of protein misassembly in yeast by oligonucleotides is analyzed.

Two S. cerevisiae strains are provided: W303-la (MAT a, Ade 2-1, trp 1-1, can 1-100, leu 2-3, 112 his 3-11, 15 ura 3-1) containing the first 170 codons of human HD with either 23 Q repeats (CAG (any of the CAG repeat may be CAA)) or 75 Q repeats, preferably fused to GFP. Each of these strains bears the insert HD gene, preferably with an NLS (nuclear localization signal), under the control of an inducible promoter (Gal 1, 10) or a constitutive promoter (GPD1). The portion of Huntingtin localizes to the nucleus and protein aggregates form in these cells. See Hughes et al., Proc. Natl. Acad. Sci. USA 98: 13201-13206 (2001), the disclosure of which is hereby incorporated by reference.

Inhibition of protein misassembly by any of the oligonucleotides described in the application, such as Kan uD3T/25G, Kan uD12T/25G, Kan uRD3/25G, or any other single stranded oligonucleotide is tested in this yeast system.

Dosage levels of the oligonucleotides are tested. In some instances, the yeast cells are treated with hydroxyurea to reduce cell growth and extend the S phase of the cell cycle. In some instances, Trichostatin A (TSA) is added prior to the addition of the oligonucleotides. TSA and oligonucleotide together could have a synergistic effect on the inhibition of protein misassembly.

Inhibition of protein misassembly is carried out by dilution of the yeast in 96-well plates containing 10³ cells per well. Huntingtin protein aggregate formation is monitored (See Hughes et al., Proc. Nat'l Acad. Sci. USA 98: 13201-13206 (2001)) using a Zeiss axiovert confocal microscope, and oligonucleotides having greatest efficacy in disrupting or inhibiting protein aggregation are identified.

All patents and publications cited in this specification are herein incorporated by reference as if each had specifically and individually been incorporated by reference herein. Although the foregoing invention has been described in some detail by way of illustration and example, it will be readily apparent to those of ordinary skill in the art, in light of the teachings herein, that certain changes and modifications may be made thereto without departing from the spirit or scope of the appended claims, which, along with their full range of equivalents, alone define the scope of invention.

1. A method for identifying, from a plurality of oligonucleotide species differing in sequence and/or composition, those oligonucleotide species that are effective to disrupt aggregation of a protein aggregant in a cell, the method comprising:
introducing each of a plurality of oligonucleotide species of disparate sequence and/or composition separately into cells that have or are likely to develop aggregation of a protein aggregant, and identifying one or more of the plurality of oligonucleotide species that is effective at preventing, reducing, or disrupting said aggregation.

2. The method of claim 1, wherein the protein aggregant is selected from the group consisting of: huntingtin, Aβ, tau, α-synuclein, atropin-1, ataxin-1, ataxin-2, ataxin-3, ataxin-7, alpha 1A, PrPSc, transthyretin, superoxide dismutase, amylin, IgG light chain, procalcitonin, β2-microglobulin, atrial natriuretic factor, serum amyloid A, apoA1, and gelsolin.

3. The method of claim 1, wherein each of the oligonucleotide species is at least 6 nt in length.

4. The method of claim 3, wherein each of the oligonucleotide species is at least 25 nt in length.

5. The method of claim 1, wherein at least a plurality of the oligonucleotide species include at least one phosphorothioate linkage.

6. The method of claim 5, wherein at least a plurality of the oligonucleotide species includes at least one phosphorothioate linkage at each terminus.

7. The method of claim 1, wherein said oligonucleotides are introduced into said cells in vitro.

8. The method of claim 1, wherein said protein aggregant is detectably labeled.

9. The method of claim 8, wherein said label is recombinantly fused to said aggregant.

10. The method of claim 12, wherein said label is a polypeptide comprising a GFP-like chromophore.

11. A method of treating a subject having a disorder caused by aggregation of a protein aggregant, the method comprising:
administering an effective amount of a composition comprising at least one oligonucleotide species that prevents, delays, or disrupts said protein aggregation, optionally in admixture with a pharmaceutically acceptable carrier or excipient.

12. The method of claim 11, wherein said disorder is selected from the group consisting of:
Alzheimer's disease, cystic fibrosis, amyotrophic lateral sclerosis, Parkinson's disease, spinobulbar muscular atrophy, spinocerebellar ataxia types 1, 2, 3, 6, and 7, dentatorubral-pallidoluysian atrophy, prion diseases, scrapie, bovine spongiform encephalopathy, CJD, new variant CJD, Pick's disease, diabetes type II, multiple myeloma-plasma cell dyscrasias, medullary carcinoma of the thyroid, chronic renal failure, congestive heart failure, chronic inflammation, atherosclerosis (apoA1), and familial amyloidosis.

13. The method of claim 11, wherein said at least one oligonucleotide species is at least 6 nt in length.

14. The method of claim 13, wherein said at least one oligonucleotide species is at least 25 nt in length.

15. The method of claim 11, wherein said at least one oligonucleotide species comprises at least one terminal modification.

16. The method of claim 15, wherein said at least one terminal modification is selected from the group consisting of: phosphorothioate linkage and 2′-O-Me analogue.

17. The method of claim 16, wherein said terminal modification is at least one phosphorothioate linkage.

18. The method of claim 11, wherein said composition comprises at least two oligonucleotide species differing in one or more of sequence, length, or composition.

19. The method of claim 11, wherein said administering comprises intravenous or intrathecal infusion.

20. The method of claim 11, wherein said administering comprises:
transfecting cells from said subject ex vivo, and then
reintroducing said transfected cells into said subject.
